(19) United States
(12) Patent Application Publication
(10) Pub. No.: US 2013/0090346 A1
(43) Pub. Date: Apr. 11, 2013

(54) Use of 1H-quinazoline-2,4-diones

(71) Applicants: Donald Johns, Woburn, MA (US); Georges Imbert, Bartenhein (FR); Klaus Kucher, Mullheim (DE)

(72) Inventors: Donald Johns, Woburn, MA (US); Georges Imbert, Bartenhein (FR); Klaus Kucher, Mullheim (DE)

(21) Appl. No.: 13/604,026

(22) Filed: Sep. 5, 2012

Related U.S. Application Data
(60) Provisional application No. 61/531,891, filed on Sep. 7, 2011.

(51) Int. Cl. C07D 239/96; C07D 403/10; A61K 45/06; C07D 405/10

(52) U.S. Cl. CPC C07D 239/96; C07D 405/10; C07D 403/10; A61K 45/06
USPC 514/266.23; 544/285; 514/266.3; 514/266.24; 544/284

(57) ABSTRACT

The invention concerns the use of competitive AMPA receptor antagonists for the treatment or prevention of photosensitive epilepsy.

### FIELD OF THE INVENTION

The present invention relates to pharmaceutical uses of 1H-quinazoline-2,4-diones, their pharmaceutically acceptable salts, and prodrugs thereof specifically for the treatment of photosensitive epilepsy (PSE).

### BACKGROUND OF THE INVENTION

Epilepsy is one of the most common neurological disorders, with lifetime prevalence in excess of 1% of the world population. Despite the fact that there are about 20 antiepileptic drugs (AEDs) on the market, there is still a high medical need for improved treatments of epilepsy since about 30-40% of patients are inadequately controlled or suffer from drug side effects.

Photosensitive epilepsy (PSE) is a rare form of reflex epilepsy in which seizures are triggered in photosensitive individuals by periodic visual stimuli such as flashing or flickering lights or regular patterns like stripes or checks. The patterns are usually high in luminance contrast (bright flashes of light alternating with darkness, or white bars against a black background). Both natural and artificial light may trigger seizures. Examples of flashing lights or rapidly changing or alternating images that may trigger the seizure in these patients include exposure to faulty lights or stroboscopic lights such as those in disco-clubs, the light of emergency vehicles, images in films or television programs (with an increased risk of seizures with closer proximity to the light source); driving at dawn or dusk through an area in which the sun is shining through a line of trees or through a sudden change in light intensity (such as coming out of a tunnel); exposure to the light pattern caused by sun flickering on water; looking out from the window of a fast moving vehicle; or observing geometric patterns.

PSE is a type of reflex epilepsy and individuals with PSE may either have seizures exclusively in response to specific stimuli, not suffer spontaneous seizures or alternatively, have reflex seizures coexisting with spontaneously occurring seizures. PSE is mostly associated with generalized epilepsies. The previously mentioned visual stimuli may provoke clinical photoconvulsive seizures or subclinical photoparoxysmal responses (PPR) in PSE patients. Photosensitive epilepsy is a generalized epileptic-form reaction on intermittent photic stimulation (IPS) outlasting the stimulus sequence in about 5% of epileptic patients. Photosensitive epilepsy has a strong genetic component and a higher incidence in females.

The triggering stimuli vary from one patient to another, as do the nature and severity of the resulting seizures (ranging from brief absence to full tonic clonic seizure). Some patients are more sensitive with their eyes closed; others are more sensitive with their eyes open.

An effective treatment for PSE is avoidance of the provoking stimulus. However this can be difficult if the real trigger is not known. Therefore, the large majority of patients with epilepsy and photosensitivity need treatment with antiepileptic drugs. The drug of choice is often Valproate in monotherapy, if necessary Clobazam could be given as an adjunctive treatment. Lamotrigine, Topiramate and Levetiracetam are recommended as second choices. Besides the rather typical adverse effects like sedation, nausea and drowsiness Valproate can cause severe liver injury particularly in patients just starting the treatment. Valproate can also cause birth defects and should not be given during pregnancy, a major problem in photosensitive epilepsy since this is mainly diagnosed in young females. Several of the above mentioned drugs also cause induction of metabolizing enzymes which can cause drug-drug interactions.

It would therefore be desirable to provide alternative or improved treatments for PSE, for example treatments not suffering from some or all of the above disadvantages/limitations.

### SUMMARY OF THE INVENTION

In accordance with a first aspect of the invention, there is provided a 1H-quinazoline-2,4-dione of formula (I)

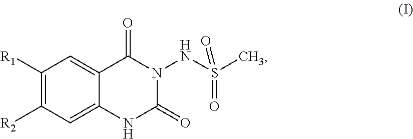

wherein
R₁ is C₁-C₆alkyl substituted by one, two or three substituents selected from hydroxy, C₁-C₆alkoxy or C₅-C₆cycloalkoxy; or
R₁ is

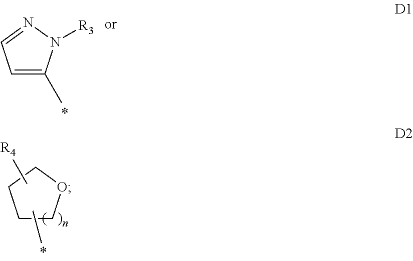

R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃fluoroalkyl;
or a pharmaceutically acceptable salt or prodrug thereof;
for use in the treatment or prevention of photosensitive epilepsy.

A second aspect of the invention concerns a method for the treatment of photosensitive epilepsy in a subject in need of such treatment, which comprises administering to said subject a therapeutically effective amount of a 1H-quinazoline-2,4-dione of formula (I) or a pharmaceutically acceptable salt or prodrug thereof.

A third aspect of the invention relates to the use of a 1H-quinazoline-2,4-dione of formula (I), or a pharmaceutically acceptable salt or prodrug thereof, for the treatment or prevention of photosensitive epilepsy.

A fourth aspect of the invention relates to a 1H-quinazoline-2,4-dione of formula (I), or a pharmaceutically acceptable salt or prodrug thereof, for the treatment or prevention of photosensitive epilepsy.

A fifth aspect of the invention relates to a pharmaceutical composition comprising a 1H-quinazoline-2,4-dione of formula (I), or a pharmaceutically acceptable salt or prodrug thereof, in the treatment or prevention of photosensitive epilepsy.

A sixth aspect of the invention relates to the use of a 1H-quinazoline-2,4-dione of formula (I), or a pharmaceutically acceptable salt or prodrug thereof, for the manufacture of a medicament for the treatment or prevention of photosensitive epilepsy.

A seventh aspect of the invention relates to a method for the treatment of photosensitive epilepsy in a subject in need of such treatment, which comprises administering to said subject a therapeutically effective amount of a 1H-quinazoline-2,4-dione of formula (I) or a pharmaceutically acceptable salt or prodrug thereof.

An eight aspect of the invention concerns a 1H-quinazoline-2,4-dione of formula (I) or a pharmaceutically acceptable salt or prodrug thereof in combination with one or more antiepileptic drugs (AEDs), preferably one or two antiepileptic drugs (AEDs), for use in the treatment or prevention of photosensitive epilepsy.

A ninth aspect of the invention relates to a formulation comprising a compound of Formula (I) e.g. Compound C7, or a pharmaceutically acceptable salt or prodrug thereof, the compound having an AUC₂₄ₕ greater than or equal to 5000 hr*ng/mL and/or Cₘₐₓ greater than or equal to 300 ng/ml e.g. such that the PPR is suppressed and/or the SPR is reduced, e.g. by at least 3 steps, optionally for use in the treatment of PSE

### BRIEF DESCRIPTION OF THE DRAWINGS

FIG. 1: Time course of mean standardized photoparoxysmal response range (SPR) for C-7 50 mg dose group.

FIG. 2: Arithmetic mean (SD) Compound C-7 concentration-time profiles following single oral administration of 15, 50 and 100 mg of Compound C-7.

FIG. 3: Time course of mean SPR and plasma PK concentrations of C-7 50 mg, PD analysis set.

FIG. 4: Time course of mean SPR and plasma PK concentrations o C-7 100 g, PD analysis set.

FIG. 5: Time course of mean SPR and plasma PK concentrations of C-7 15 mg, PD analysis set.

### DETAILED DESCRIPTION OF THE INVENTION

The invention relates to a 1H-quinazoline-2,4-dione of formula (I)

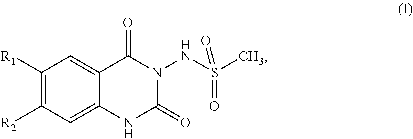

wherein
R₁ is C₁-C₆alkyl substituted by one, two or three substituents selected from hydroxy, C₁-C₆alkoxy or C₅-C₆cycloalkoxy; C₅-C₆cycloalkyl substituted by one two or three substituents selected from hydroxy, C₁-C₆alkoxy or C₅-C₆cycloalkoxy; or
R₁ is

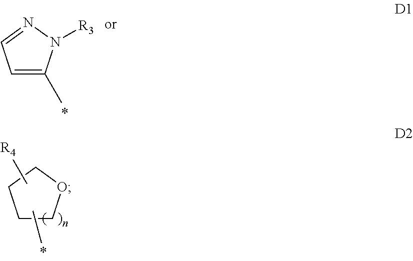

R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃fluoroalkyl;
their pharmaceutically acceptable salts, and their prodrugs thereof;
for use in a method for the treatment or prevention of photosensitive epilepsy.

The compound of formula (I) is a competitive AMPA antagonist. It is well understood that allosteric (non-competitive) antagonists provide an insurmountable blockade of AMPA receptors, potentially preventing any AMPA receptor-mediated neurotransmission at the synapse. In contrast, a high concentration of glutamate at the synapse can still activate the post-synaptic membrane in the presence of a competitive AMPA antagonist (albeit with a lower efficacy). Competitive AMPA antagonists may therefore exhibit an improved safety profile, as they will not fully block neurotransmission, but instead reduce the exaggerated glutamate signaling observed in some neurological disease, e.g. epilepsy.

Compounds of the formula (I) not only block AMPA-induced glutamate release from activated astrocytes but after oral dosing also suppress epilepsy seizures in epilepsy or in Rasmussen encephalitis.

The compound of the invention of formula (I) in addition to the advantage of being a competitive AMPA antagonist receptor inhibitor, presents also the advantage of being a selective competitive AMPA antagonist. Furthermore the compound of the invention of formula (I) is capable of penetrating the blood brain barrier and may be formulated in an oral dosage form.

In the present specification, the following definitions shall apply if no specific other definition is given:

Bonds with the asterisk (*) denote point of binding to the rest of the molecule.

“C₁-C₆alkyl ” represents a straight-chain or branched-chain alkyl group; for example, methyl, ethyl, n- or iso-propyl, n-, iso-, sec- or tert-butyl, n-pentyl, n-hexyl, with particular preference given to methyl, ethyl, n-propyl and iso-propyl.

“C₅-C₆cycloalkyl” represents cyclopentyl or cyclohexyl; preferably cyclopentyl.

Each alkyl/cycloalkyl-part of “alkoxy”, “cycloalkoxy”, “alkoxyalkyl” and “fluoroalkyl” shall have the same meaning as described in the above-mentioned definitions of “alkyl”/“cycloalkyl”.

“C₁-C₃fluoroalkyl” preferably represents trifluoromethyl, difluoromethyl or fluoromethyl.

It will be understood that any discussion of methods or references to the active ingredients includes said active ingredient in free form and in form of a pharmaceutically acceptable salt. If the active ingredients have for example, at least one acidic center (for example COOH) they can form salts with bases. The active ingredient or a pharmaceutically acceptable salt thereof may also be used in the form of a hydrate or may include other solvents used for crystallization.

A “pharmaceutically acceptable salt” is intended to mean a salt of a free base/free acid of a compound represented by formula (I) that is not toxic, biologically intolerable, or otherwise biologically undesirable. Preferred pharmaceutically acceptable salts are those that are pharmacologically effective and suitable for contact with the tissues of patients without undue toxicity, irritation, or allergic response. Such salts are known in the field (e.g. S. M. Berge, et al, “Pharmaceutical Salts”, J. Pharm. Sd., 1977, 66:1-19; and “Handbook of Pharmaceutical Salts, Properties, Selection, and Use”, Stahl, R H., Wermuth, C. G., Eds.; Wiley-VCH and VHCA: Zurich, 2002).

In one embodiment of the invention, the ¹H-quinazoline-2,4-diones of formula (I) is used in free form.

The 1H-quinazoline-2,4-diones of formula (I), their manufacture and their use as competitive AMPA receptor antagonists are known from WO 2006/108591 or can be prepared analogously to said reference. WO 2006/108591 is incorporated herein by reference.

On account of asymmetrical carbon atom(s) that may be present in the 1H-quinazoline-2,4-diones of formula (I) and their pharmaceutically acceptable salts, the compounds may exist in optically active form or in form of mixtures of optical isomers, e.g. in form of racemic mixtures or diastereomeric mixtures. All optical isomers and their mixtures, including racemic mixtures, are part of the present invention.

In one embodiment of the invention, the 1H-quinazoline-2,4-dione of formula (I) is a compound, wherein R₁ is C₁-C₆alkyl substituted by one two or three substituents selected from hydroxy, C₁-C₆alkoxy or C5-C₆cycloalkoxy; and R₂ is C₁-C₃alkyl or C₁-C₃fluoroalkyl.

In one embodiment of the invention, the 1H-quinazoline-2,4-dione of formula (I) is a compound, wherein R₁ is

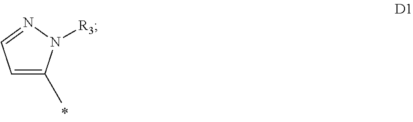

R₃ is C₁-C₆alkyl, hydroxy or C₁-C₆alkoxy-C₁-C₆alkyl; and R₂ is C₁-C₃alkyl or C₁-C₃fluoroalkyl.

In one embodiment of the invention, the 1H-quinazoline-2,4-dione of formula (I) is a compound, wherein R₁ is

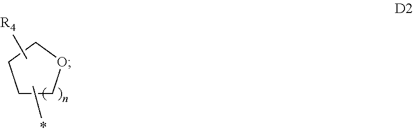

R₄ is hydrogen or C₁-C₆alkyl; n is 1 or 2; and R₂ is C₁-C₃alkyl or C₁-C₃fluoroalkyl.

In one embodiment of the invention, the 1H-quinazoline-2,4-dione of formula (I) is a compound selected from the group consisting of
A-1: N-[6-(1-Hydroxy-ethyl)-2,4-dioxo-7-trifluoromethyl-1,4-dihydro-2H-quinazolin-3-yl]-methanesulfonamide;
A-2: N-[6-(1-Methoxy-ethyl)-2,4-dioxo-7-trifluoromethyl-1,4-dihydro-2H-quinazolin-3-yl]-methanesulfonamide;
A-3: N-[6-(1-Hydroxy-propyl)-2,4-dioxo-7-trifluoromethyl,-1,4-dihydro-2H-quinazolin-3-yl]-methanesulfonamide;
A-4: N-[6-(1-Isopropoxy-ethyl)-2,4-dioxo-7-trifluoromethyl-1,4-dihydro-2H-quinazolin-3-yl]-methanesulfonamide;
A-5: N-[6-(1-Ethoxy-ethyl)-2,4-dioxo-7-trifluoromethyl-1,4-dihydro-2H-quinazolin-3-yl]-methanesulfonamide;
A-6: N-[2,4-Dioxo-6-(1-propoxy-propyl)-7-trifluoromethyl-1,4-dihydro-2H-quinazolin-3-yl]-methanesulfonamide;
A-7: N-[6-(1-isopropoxy-propyl)-2,4-dioxo-7-trifluoromethyl-1,4-dihydro-2H-quinazolin-3-yl]-methanesulfonamide;
A-8: N-[7-Difluoromethyl-6-(1-ethoxy-ethyl)-2,4-dioxo-1,4-dihydro-2H-quinazolin-3-yl]-methanesulfonamide;
A-9: N-[2,4-Dioxo-6-(1-propoxy-ethyl)-7-trifluoromethyl-1,4-dihydro-2H-quinazolin-3-yl]-methanesulfonamide;
A-10: N-[6-(1-Butoxy-ethyl)-2,4-dioxo-7-trifluoromethyl-1,4-dihydro-2H-quinazolin-3-yl]-methanesulfonamide;
A-11: N-[6-(1-Isobutoxy-ethyl)-2,4-dioxo-7-trifluoromethyl-1,4-dihydro-2H-quinazolin-3-yl]-methanesulfonamide;
A-12: N-[6-(1-methoxy-butyl)-2,4-dioxo-7-trifluoromethyl-1,4-dihydro-2H-quinazolin-3-yl]-methanesulfonamide;
A-13: N-[6-(1-Ethoxy-propyl)-2,4-dioxo-7-trifluoromethyl-1,4-dihydro-2H-quinazolin-3-yl]-methanesulfonamide;
A-14: N-[6-(1-Cyclopentyloxy-ethyl)-2,4-dioxo-7-trifluoromethyl-1,4-dihydro-2H-quinazolin-3-yl]-methanesulfonamide;
A-15: N-[6-(1-Hydroxy-butyl)-2,4-dioxo-7-trifluoromethyl-1,4-dihydro-2H-quinazolin-3-yl]-methanesulfonamide;
A-16: N-[6-(1-Methoxy-2-methyl-propyl)-2,4-dioxo-7-trifluoromethyl-1,4-dihydro-2H-quinazolin-3-yl]-methanesulfonamide;
A-17: N-[6-(3-Hydroxy-propyl)-2,4-dioxo-7-trifluoromethyl-1,4-dihydro-2H-quinazolin-3-yl]-methanesulfonamide;
A-18: N-[6-(1-Hydroxy-3-methoxy-propyl)-2,4-dioxo-7-trifluoromethyl-1,4-dihydro-2H-quinazolin-3-yl]-methanesulfonamide;
A-19: N-[6-(1-Hydroxy-2-methyl-propyl)-2,4-dioxo-7-trifluoromethyl-1,4-dihydro-2H-quinazolin-3-yl]-methanesulfonamide;
B-1: N-[2,4-Dioxo-6-(tetrahydro-pyran-2-yl)-7-trifluoromethyl-1,4-dihydro-2H-quinazolin-3-yl]-methanesulfonamide;
B-2: N-[2,4-Dioxo-6-(tetrahydro-furan-2-yl)-7-trifluoromethyl-1,4-dihydro-2H-quinazolin-3-yl]-methanesulfonamide;
B-3: N-[2,4-Dioxo-6-(tetrahydro-furan-3-yl)-7-trifluoromethyl-1,4-dihydro-2H-quinazolin-3-yl]-methanesulfonamide;
C-1: N-{7-Isopropyl-6-[2-(2-methoxy-ethyl)-2H-pyrazol-3-yl]-2,4-dioxo-1,4-dihydro-2H-quinazolin-3-yl}-methanesulfonamide;
C-2: N-[6-(2-Isopropyl-2H-pyrazol-3-yl)-2,4-dioxo-7-trifluoromethyl-1,4-dihydro-2H-quinazolin-3-yl]-methanesulfonamide;
C-3: N-[7-Fluoromethyl-6-(2-isopropyl-2H-pyrazol-3-yl)-2,4-dioxo-1,4-dihydro-2H-quinazolin-3-yl]-methanesulfonamide;
C-4: N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;
C-5: N-[6-(2-Hydroxy-2H-pyrazol-3-yl)-2,4-dioxo-7-trifluoromethyl-1,4-dihydro-2H-quinazolin-3-yl]-methanesulfonamide;
C-6: N-[7-Ethyl-6-(2-isopropyl-2H-pyrazol-3-yl)-2,4-dioxo-1,4-dihydro-2H-quinazolin-3-yl]-methanesulfonamide;
C-7: N-[7-Isopropyl-6-(2-methyl-2H-pyrazol-3-yl)-2,4-dioxo-1,4-dihydro-2H-quinazolin-3-yl]-methanesulfonamide;
C-8: N-[7-Isopropyl-6-(2-isopropyl-2H-pyrazol-3-yl)-2,4-dioxo-1,4-dihydro-2H-quinazolin-3-yl]-methanesulfonamide;
C-9: N-[7-Difluoromethyl-6-(2-methyl-2H-pyrazol-3-yl)-2,4-dioxo-1,4-dihydro-2H-quinazolin-3-yl]-methanesulfonamide;
C-10: N-[7-Difluoromethyl-6-(2-isopropyl-2H-pyrazol-3-yl)-2,4-dioxo-1,4-dihydro-2H-quinazolin-3-yl]-methanesulfonamide;
C-11: N-[7-Ethyl-6-(2-methyl-2H-pyrazol-3-yl)-2,4-dioxo-1,4-dihydro-2H-quinazolin-3-yl]-methanesulfonamide;
C-12: N-[7-Ethyl-6-(2-ethyl-2H-pyrazol-3-yl)-2,4-dioxo-1,4-dihydro-2H-quinazolin-3-yl]-methanesulfonamide;
C-13: N-[7-Fluoromethyl-6-(2-methyl-2H-pyrazol-3-yl)-2,4-dioxo-1,4-dihydro-2H-quinazolin-3-yl]-methanesulfonamide;
C-14: N-[7-(1-fluoro-ethyl)-6-(2-methyl-2H-pyrazol-3-yl)-2,4-dioxo-1,4-dihydro-2H-quinazolin-3-yl]-methanesulfonamide;
C-15: N-[7-(1,1-difluoro-ethyl)-6-(2-methyl-2H-pyrazol-3-yl)-2,4-dioxo-1,4-dihydro-2H-quinazolin-3-yl]-methanesulfonamide;
C-16: N-[7-(1,1-difluoro-ethyl)-6-(2-isopropyl-2H-pyrazol-3-yl)-2,4-dioxo-1,4-dihydro-2H-quinazolin-3-yl]-methanesulfonamide;
C-17: N-[7-(1-fluoro-ethyl)-6-(2-isopropyl-2H-pyrazol-3-yl)-2,4-dioxo-1,4-dihydro-2H-quinazolin-3-yl]-methanesulfonamide; and
C-18: N-[6-(2-Methyl-2H-pyrazol-3-yl)-2,4-dioxo-7-trifluoromethyl-1,4-dihydro-2H-quinazolin-3-yl]-methanesulfonamide.

The compounds of the invention, including the specific exemplified compounds may be prepared by any suitable method, e.g. as described in WO 2006/108591.

In one embodiment of the invention, the 1H-quinazoline-2,4-dione of formula (I) is a compound selected from the group consisting of compound A-1, A-2, A-3, A-4, A-5, A-6, A-7, A-8, A-9, A-10, A-11, A-12, A-13, A-14, A-15, A-16, A-17, A-18 and A-19.

In one embodiment of the invention, the 1H-quinazoline-2,4-dione of formula (I) is a compound selected from the group consisting of compound B-1, B-2 and B-3.

In one embodiment of the invention, the 1H-quinazoline-2,4-dione of formula (I) is a compound selected from the group consisting of compound C-1, C-2, C-3 C-4, C-5, C-6, C-7, C-8, C-9, C-10, C-11, C-12, C-13, C-14, C-15, C-16, C-17 and C-18.

Advantageous compounds of the invention, i.e., the 1H-quinazoline-2,4-diones of formula (I), should be well absorbed from the gastrointestinal tract, penetrate the blood brain barrier, be sufficiently metabolically stable and possess favorable pharmacokinetic properties.

Preferred compounds, having superior bioavailability are 1H-quinazoline-2,4-dione of formula (I) selected from the group consisting of compounds: A-1, A-2, A-3, A-4, A-5, A-6, A-7, A-13, A-14, A-15, A-18, B-2, B-3, C-1, C-2, C-3, C-4, C-5, C-6, C-7, C-8, C-9, C-10, C-11, C-12 C-15, C-16, C-17 and C-18.

More preferred compounds, having, superior bioavailability are 1 H-quinazoline-2,4-dione of formula (I) selected from the group consisting of compounds: A-1, A-2, A-3, A-4, A-5, A-7, A-15, B-2, B-3, C-1 C-2, C-3, C-6, C-7, C-8, C-9, C-10, C-11, C-12, C-15, C-17 and C-18.

Further more preferred compounds, having superior bioavailability are 1 H-quinazoline-2,4-dione of formula (I) selected from the group consisting of compounds: A-2, A-3, A-4, A-5 B-2, C-2, C-3, C-7, C-9, C-10, C-11, C-15 and C-18.

Most preferred compounds, having superior bioavailability are 1H-quinazoline-2,4-dione of formula (I) selected from the group consisting of compounds: A-2, A-5, B-2, C-7, C-9 and C-11, such as compound C-7.

Compounds for use in the present invention are either obtained in the free form, as a salt thereof, or as prodrug derivatives thereof.

The term “prodrug” as used herein relates to a compound, which converts in vivo into a compound used in the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. The term “prodrug,” as used herein, represents in particular compounds which are transformed in vivo to the parent compound, for example, by hydrolysis in blood, for example as described in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), and “The Organic Chemistry of Drug Design and Drug Action”, 2^{nd} Edition, R B Silverman (particularly Chapter 8, pages 497 to 557), Elsevier Academic Press, 2004.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. alkyl esters |
| Alcohol | Esters, including e.g. sulfates and phosphates as |
| | well as carboxylic acid esters |
| Amine | Amides, carbamates, imines, enamines, Carbonyl |
| | (aldehyde, Imines, oximes, acetals/ketals, enol |
| | esters, ketone) oxazolidines and thiazoxolidines |

Prodrugs also include compounds convertible to the active drug by an oxidative or reductive reaction. As examples may be mentioned

### Oxidative Activation

N- and O-dealkylation
Oxidative deamination
N-oxidation
Epoxidation

### Reductive Activation

Azo reduction
Sulfoxide reduction
Disulfide reduction
Bioreductive alkylation
Nitro reduction.

Each of the above described reactions and/or reaction steps can be used individually or in combination in a method to prepare a AMPA-inhibitor or a prodrug thereof.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like.

The term “hydrate” refers to the complex where the solvent molecule is water. The compounds of the present invention, including salts, hydrates and solvates thereof, may inherently or by design form polymorphs.

Preferred prodrugs of the invention should be well absorbed from the gastrointestinal tract, be transformed into the parent compound (or active principle, being the compound that in-vivo acts as AMPA receptor antagonist), the parent compound should be sufficiently metabolically stable and possess favorable pharmacokinetic properties.

Further preferred prodrugs of the invention lead to an oral bioavailability of the parent compound which is comparable to the bioavailability when administered as a drug.

Further preferred prodrugs of the invention exhibit increased oral bioavailability compared to the parent compound when administered as a drug. Oral bioavailability may manifest itself in different ways: (i) a biological effect may be achieved after oral administration when the parent compound is less effective upon oral administration, (ii) an earlier onset of action upon oral administration, (iii) a lower dose needed to achieve the same effect, (iv) a higher effect achieved by the same dose or (v) a prolonged action at the same dose.

Further preferred prodrugs of the invention are transformed into parent compounds which in-vivo bind potently to AMPA receptors whilst showing little affinity for other receptors.

Some prodrugs of the invention are transformed into parent compounds which also show antagonistic activity at kainate receptors. Besides such dual activity, showing little affinity for other receptors is a preferred feature.

Further prodrugs of the invention—when the active principle is targeted against receptors in the central nervous system—are transformed into parent compounds that cross the blood brain barrier freely.

Further prodrugs of the invention—when the active principle is targeted selectively against receptors in the peripheral nervous system—are transformed into parent compounds that do not cross the blood brain barrier.

Prodrugs, parent compounds and released pro-moieties should be non-toxic and demonstrate few side-effects.

Furthermore, the ideal prodrug of the invention will be able to exist in a physical form that is stable, non-hygroscopic and easily formulated.

The higher oral bioavailability of the compounds for use in the invention may give rise to the following beneficial effects relating to less bioavailable compounds: (i) an enhanced biological effect may be achieved after oral administration; (ii) an earlier onset of action may be observed following oral administration; (iii) a lower dose may be needed to achieve the same effect; (iv) a higher effect may be achieved by the same dose or (v) a prolonged action may be observed at the same dose.

Preferably the compound for use in the invention when tested in-vivo potently binds to AMPA receptors whilst showing little affinity for other receptors.

The term “subject” as used herein typically refers to a mammal, e.g. a human, especially to a human patient diagnosed with photosensitive epilepsy (PSE).

The term “treatment” as used herein refers to any type of treatment that imparts a benefit to a subject affected with photosensitive epilepsy, e.g. a human patient diagnosed with PSE, including prevention or reduction in number and severity of seizures.

The term “therapeutically effective amount” as used herein typically refers to a drug amount which, when administered to a subject, is sufficient to provide a therapeutic benefit, e.g. is sufficient for treating or preventing the photosensitive epileptic seizure (e.g. the amount provides an amelioration of symptoms, e.g. it leads to a reduction in number and severity of seizures).

For the above-mentioned indications (the conditions and disorders) the appropriate dosage will vary depending upon, for example, the compound employed, the host, the mode of administration and the nature and severity of the condition being treated. However, in general, satisfactory results in animals are indicated to be obtained at a daily dosage of from about 0.01 to about 100 mg/kg body weight, preferably from about 1 to about 30 mg/kg body weight, e.g. 10 mg/kg. In larger mammals, for example humans, an indicated daily dosage is in the range from about 0.1 to about 1000 mg, preferably from about 1 to about 400 mg, most preferably from about 10 to about 100 mg of a 1H-quinazoline-2,4-dione of formula (I) conveniently administered, for example, in divided doses up to four times a day. In one embodiment, about 100 mg of a 1H-quinazoline-2,4-dione of formula (I) is administered daily. In a further embodiment, about 200 mg of a 1H-quinazoline-2,4-dione of formula (I) is administered daily.

For use according to the invention, the 1H-quinazoline-2,4-diones of formula (I) may be administered as single active agent or in combination with one or more other active agents, in any usual manner, e.g. orally, for example in the form of tablets, capsules or drinking solutions; rectally, for example in the form of suppositories; intravenously, for example in the form of injection solutions or suspensions; or transdermally, for example in the form of a patch.

In one embodiment, the manner of administration is oral administration, for example in the form of a tablet, capsule or drinking solution. In one embodiment, the manner of administration is rectal administration, for example in the form of a suppository. In one embodiment, the manner of administration is transdermal administration, for example in the form of a patch. In one preferred embodiment, the manner of administration is oral administration.

Preferred pharmaceutical compositions comprise a 1H-quinazoline-2,4-dione of formula (I) in association with at least one pharmaceutical carrier or diluent. Such compositions may be manufactured in conventional manner. Unit dosage forms may contain the compound of formula (I) e.g. compound C7 in an amount greater than or equal to 2.5 mg, for example greater than or equal to 5 mg such as for example greater than or equal to 10 mg or for example greater than or equal to 15 mg. Unit dosage forms may also contain the compound of formula (I) e.g. compound C7 in an amount of greater than or equal to 40 mg, 50 mg, 75 mg or 100 mg or greater than or equal to 150 mg or 200 mg.

Unit dosage forms may contain the compound of formula (I) e.g. compound C7 in an amount less than or equal to 300 mg, for example less than or equal to 200 mg, such as for example less than or equal to 150 mg or for example less than or equal to 100 mg.

Unit dosage forms may also contain the compound of formula (I) e.g. compound C7 in an amount in the range from 5-200 mg e.g. 10-150 mg or 15-100 mg such as 50-100 mg.

The pharmaceutical compositions according to the invention are compositions for enteral administration, such as oral or rectal administration; or parenteral administration, such as intramuscular, intravenous, and nasal or transdermal administration, to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be for example, in unit dose form, such as in the form of ampoules, vials, suppositories, dragées, tablets or capsules.

The pharmaceutical compositions of the present invention may be, prepared in a manner known per se, for example by means of conventional dissolving, lyophilizing, mixing, granulating or confectioning processes. Such processes are exemplified in WO 2005/079802, WO 2003/047581, WO 2004/000316, WO 2005/044265, WO 2005/044266 WO 2005/044267, WO 2006/114262 and WO 2007/071358.

The compound of formula (I) e.g. C7, which is comprised in the composition, may give an AUC₂₄ₕ or Cₘₐₓ such that the PPR is suppressed and/or the SPR is reduced, e.g. by at least 3 steps.

The compound of formula (I) e.g. C7, which is comprised in the composition, may give an AUC₂₄ₕ greater than or equal to 5000 hr*ng/mL, for example greater than or equal to 8000 hr*ng/mL, such as greater than or equal to 10000 hr*ng/mL or greater than or equal to 12000 hr*ng/mL or 15000 hr*ng/mL. The compound of formula (I) e.g. C7, which is comprised in the composition, may also give an AUC₂₄ₕ less than or equal to 25000 hr*ng/mL, for example less than or equal to 22000 hr*ng/mL, such as less than or equal to 20000 hr*ng/mL.

Alternatively or in addition, the compound of formula (I) e.g. compound C7 which is comprised in the composition may give a Cₘₐₓ of greater than or equal to 300 ng/mL, such as greater than or equal to 400 ng/mL or greater than or equal to 500 ng/mL e.g greater than or equal to 750 ng/mL, greater than or equal to 1000 ng/mL, greater than or equal to 1400 ng/mL, greater than or equal to 1800 ng/mL or greater than or equal to 2400 ng/mL such as greater than or equal to 2800 ng/mL. The compound of formula (I) e.g. compound C7 which is comprised in the composition may also give a Cₘₐₓ of less than or equal to 4000 ng/mL, such as less than or equal to 3500 ng/mL or less than or equal to 3000 ng/mL.

It will be understood that, for a given formulation, some variation in AUC₂₄ₕ or Cₘₐₓ may be observed from patient to patient. The skilled person would understand that in this situation AUC₂₄ₕ or Cₘₐₓ is an aggregate value obtainable using a meaningful patient sample size e.g. as defined in EU or US clinical guidelines as in force at the filing date.

Compositions for transdermal administration are described in Remington's Pharmaceutical Sciences 16^{th} Edition Mack; Sucker, Fuchs and Spieser, Pharmazeutische Technologie, 1^{st} Edition, Springer.

### 1. Diagnosis of Photosensitive Epilepsy

Epilepsy is usually diagnosed by observing seizures occurring spontaneously. A seizure is a burst of abnormal electrical activity in the brain. Seizures can originate in a single location and involve a relatively small area (partial seizures), or they may involve the entire brain (generalized seizures). Certain epilepsy syndromes require particular precipitants or triggers for seizures to occur. Such syndromes are termed reflex epilepsy. Diagnosis of Photosensitive Epilepsy may be made by noting a correlation between the exposure to specific visual stimuli and seizure activity. This may be straightforward in situations where the seizures may impair the PSE subject's everyday life, e.g. by limiting his ability to drive a car. In other cases, patients suffering from PSE often are not recognized as affected by PSE as it is not always easy to identify that they are undergoing photosensitive epileptic episodes. Some seizures are so subtle that they can easily go unnoticed by the person experiencing them or by others. While the seizures themselves may not be noticed, the after-effects may include disabilities that linger for days: difficulties with mood, focus, memory, learning, sleep, sensory perception, and other functions.

Clinical methods for diagnosing photosensitive epilepsy are known. Photosensitive epilepsy is a reflex form of epilepsy, in which epileptiform electroencephalogram (EEG) discharges may be evoked at any time by intermittent photic stimulation (IPS). This EEG response is also called photoparoxysmal response (PPR).

Each patient displays a different photosensitivity range, which is the difference between the lower and upper sensitivity limit to the visual stimulation. This range is related to liability of seizures in daily life. The sensitivity range is specific for each patient and can be modified or abolished by using antiepileptic medicaments.

In patients with photosensitive epilepsy, a standardized photoparoxysmal response range (SPR) may be determined by performing the IPS with a standardized series of flash frequencies, for instance 14 frequencies from a range of 2 to 60 Hz. SPR is a dimensionless parameter and is defined as the number of frequencies (steps) between the lowest and highest frequencies, e.g. between 2 and 60 Hz that consistently elicit a PPR. If 14 frequencies are tested, the minimum and maximum possible SPR values are 0 and 14. Zero means complete abolition of reactivity on any stimulation frequency, i.e. complete abolishment of the PPR. SPR is relatively stable for each patient and reflects the liability for seizures in daily life. The potential efficacy of an AED could be assessed by measuring hourly changes in the SPR after a single oral dose given to the PSE patients.

The efficacy of the compounds of formula (I) in the treatment and prevention of PSE is confirmed by the following worked examples.

### EXAMPLES

The compound C-7 used in the study described hereinafter is an orally active compound of general formula (I).

The study used the IPS/PPR paradigm. It was a multicenter, non-randomized, single blind, within-subject, placebo-controlled proof of concept study conducted to assess the effect of single oral doses of the compounded formula C-7 in suppressing the photoparoxysmal response (PPR) or reducing the standardized photoparoxysmal response range (SPR) in patients with PSE. Photosensitivity was detected using electroencephalography (EEG) measurements of subjects exposed to Intermittent Photic Stimulation (IPS). This EEG response was a photoparoxysmal response (PPR). For the purpose of the study, provocation of PPRs by IPS, not of seizures, was required. The SPR of each patient was the number of standard visual stimulation frequencies (in Hertz) that the patient was sensitive to between the lower and upper thresholds. 14 frequencies were tested (from 2 to 60 Hertz). The time of onset and the duration of response (PPR suppression or SPR reduction) to the treatment with Compound C-7 in patients with PSE and the maximal reduction in SPR in patients with PSE were also evaluated.

Moreover in, this study the pharmacokinetic profile of Compound C-7 in patients with PSE was measured.

### Study

### 1. Demographic and Other Baseline Characteristics

The study included 6 patients in Cohort I, 4 patients in Cohort II and 3 patients in Cohort III. Of these, 3 patients participated twice i.e. participated in two cohorts after the 3-month washout period; and therefore a total of 10 patients participated in the entire study.

TABLE 1
Demographic and Background Characteristics

| | | Cohort I | Cohort II | Cohort III | |
|---|---|---|---|---|---|
| | | C-7 50 mg | C-7 100 mg | C-7 15 mg | Total |
| | | N = 6 | N = 4 | N = 3 | N = 13 |
| Age (years) | Mean (SD) | 28.0 (10.94) | 24.0 (2.31) | 40.7 (21.13) | 29.7 (12.96) |
| | Median | 23.5 | 24.0 | 38.0 | 25.0 |
| | Range | (20, 48) | (22, 26) | (21, 63) | (20, 63) |
| Gender - n | Male | 1 | 0 | 2 | 3 |
| | Female | 5 | 4 | 1 | 10 |
| Race - n | Caucasian | 6 | 4 | 3 | 13 |
| Ethnicity - n | Other | 6 | 4 | 3 | 13 |
| Weight (kg) | Mean (SD) | 73.08 (12.607) | 73.30 (16.556) | 77.07 (10.132) | 74.07 (12.441) |
| | Median | 72.70 | 74.55 | 78.00 | 77.00 |
| | Range | (57.0, 92.6) | (52.0, 92.1) | (66.5, 86.7) | (52.0, 92.6) |
| Height (cm) | Mean (SD) | 171.3 (7.06) | 168.3 (7.89) | 178.0 (16.52) | 171.9 (9.78) |
| | Median | 172.5 | 165.0 | 177.0 | 172.0 |
| | Range | (163, 180) | (163, 180) | (162, 195) | (162, 195) |
| BMI (kg/m²) | Mean (SD) | 24.856 (3.7356) | 25.748 (4.5592) | 24.583 (4.5190) | 25.067 (3.8275) |
| | Median | 23.848 | 27.454 | 22.801 | 25.895 |
| | Range | (21.45, 29.73) | (19.10, 28.98) | (21.23, 29.72) | (19.10, 29.73) |

BMI = body mass index; SD = standard deviation; n = patients with non-missing data points; and N = analysis set total.

The selected patients had a documented diagnosis of epilepsy for at least 6 months prior to initial dosing of compound C-7. The selected patients showed a consistent PPR during EEG evaluations prior to the first dosing indicating a diagnosis of photosensitive epilepsy. Women of child-bearing potential (WOCBP) were asked to use acceptable methods of contraception. Patients with an SPR value of 3 or less at the screening and patients with no consistent PPR (less than 3 SPR steps difference between the two evaluations required) when stimulated by intermittent photic stimulation (IPS), as determined by comparison between screening and pre-dose Day 1, were excluded from the study. Furthermore subjects with history of status epilepticus and/or regular use of benzodiazepines as rescue medication, subjects with evidence or history of any medically significant cardiac, respiratory hepatic, gastrointestinal, renal, hematologic, oncologic or progressive neurological disorder, requiring current medical intervention/therapy likely to have a significant impact on the outcome of this study, were also excluded.

There were three periods: a screening period for the assessment of patient's eligibility for participation in the study; a 3-day in hospital treatment period with single-blind dosing with placebo on days 1 and 3, and the compound of formula C-7 on day 2; and a study completion period consisting of a follow-up call by the medical investigator to the patient between day 10 and day 14 inclusive and a full study completion visit between day 29 and day 33 inclusive.

The dose of the compound of formula C-7 in Cohort studies I, II and III was 50, 100 and 15 mg respectively.

Some patients in Cohort I were treated with one concomitant AED from a pre-selected list of AEDs (valproate, lamotrigine, levetiracetam, clobazam, topiramate, pregabalin, gabapentin, zonisamide). Some patients in Cohort II and III were treated with a combination of two concomitant AEDs from a preselected list of AEDs devoid of interaction potential with the compound of formula C-7 (valproate, lamotrigine, levetiracetam, clobazam, topiramate, zonisamide). A stable dosing regimen was required for at least 4 weeks prior to initial dosing, and throughout the entire study.

With the exception of the study drug, only medication required to treat AEs was permitted from the start of screening until the end of all evaluations.

### 2. Antiepileptic Drugs (AEDs)

Some patients were treated with one or a combination of maximum two concomitant AEDs (cohorts II and III only), but had to be on stable dosing regimen for at least 4 weeks prior to initial dosing, and throughout the entire study.

The allowed AEDs were: valproate, lamotrigine levetiracetam, clobazam, topiramate and zonisamide

With the exception of two patients in Cohort I and one patient in Cohort III, who did not take AEDs, patients had pre-existing treatment with 1 AED (lamotrigine valproate or zonisamide) or 2 AEDs (levetiracetam and topiramate for one patient in Cohort III). It was noted that one of the patients of Cohort I also participated in Cohort III, and therefore in total only 2 patients did not have prior concomitant AEDs in the study. Similar AEDs concentration-time profiles were observed on Days 1, 2 and 3 for each patient. Two of the patients in Cohort I were also enrolled in Cohort II. Their lamotrigine PK profiles were similar in the 2 treatment periods. These observations confirm stable pre-existing treatment for all the patients. The co-administration of C-7 on Day 2 did not seem to affect the AED PK, since no difference in the PK profiles could be observed between Day 1 and Days 2 and 3.

A list of patients with concomitant AEDs and their PD effect is presented in Table 2. Of the 9 patients who showed suppression of PPR, 5 patients received lamotrigine (3 in C-7 50 mg group, 2 in C-7 100 mg group), 2 patients did not receive any AED (1 each in C-7 50 mg and 15 mg groups), and 1 patient each received valproate (C-7 100 mg group) and a combination of levetiracetam and topiramate (C-7 15 mg group). Moreover, all 13 patients, either receiving a concomitant AED or not showed reduction of SPR (≧3) in at least one eye condition after administration of C-7. Overall, it was noted that concomitant treatment with selected AEDs did not impact the PD effect of C-7.

TABLE 2
List of patients with AEDs and their PD effect of Compound C7

| | | |
|---|---|---|
| Center/Patient # | Concomitant AEDs | PD effect of C7^{d} |

Cohort I, C-7 50 mg

| | | |
|---|---|---|
| Patient A^{a} | Lamotrigine 100 mg bid | Suppression of PPR |
| Patient B | Valproate 500 mg bid | Reduction of SPR |
| | | (≧3) |
| Patient C | None | Reduction of SPR |
| | | (≧3) |
| Patient D | Lamotrigine 400 mg | Suppression of PPR |
| Patient E^{b} | Lamotrigine 175; 150 mg | Suppression of PPR |
| Patient F^{c} | None | Suppression of PPR^{e} |

Cohort II, C-7 100 mg

| | | |
|---|---|---|
| Patient G^{a} | Lamotrigine 100 mg bid | Suppression of PPR |
| Patient H^{b} | Lamotrigine 175; 150 mg | Suppression of PPR |
| Patient I | Zonisamide 100 mg bid | Reduction of SPR |
| | | (≧3) |
| Patient L | Valproate 300 mg bid | Suppression of PPR |

Cohort III, C-7 15 mg

| | | |
|---|---|---|
| Patient M | Levetiracetam 1000; 1500 mg | Suppression of PPR^{e} |
| | Topiramate 100 mg | |
| Patient N | Valproate 600 mg bid | Reduction of SPR |
| | | (≧3) |
| Patient O^{c} | None | Suppression of PPR |

^{a,b,c} Patients enrolled in two cohorts.
^{d}All patients showed the PD effect of reduction of SPR (≧3) in at least one eye condition after administration of C-7.
^{e}Suppression of PPR was noted already at “0” hours of dosing on Day 2.

### 3. Pharmacokinetic and Pharmacodynamic (PK/PD) Measurements

Data sets for analysis were grouped by cohorts. All 13 patients enrolled in the study were included in PK, PD, and safety analysis sets

### 3.1 Pharmacokinetics (PK)

The C-7 PK parameters were determined in plasma using non-compartmental methods using WinNonlin Pro (Version 5.2), as detailed in Table 3.

TABLE 3
Compound C-7 PK parameters determined

| | |
|---|---|
| AUCₗₐₛₜ | The area under the concentration-time curve from time zero |
| | to the last measurable concentration sampling time [ng/mL · h] |
| AUC₀₋ₜ | The area under the plasma concentration-time curve from time |
| | zero to concentration sampling time (t) [ng/mL · h] |
| AUC_{inf} | The AUC from time zero to infinity [mass × time × volume − 1] |
| Cₘₐₓ | The maximum (peak) observed drug concentration over a dosing |
| | interval at steady-state [ng/mL] |
| Tₘₐₓ | The time to reach maximum (peak) plasma drug concentration |
| | [h] |
| T_{lag} | Lag-time [h] |
| T_{1/2} | The elimination half-life associated with the terminal slope (λz) |
| | of a semi logarithmic concentration-time curve [time]. Use |
| | qualifier for other half-lives |

Biofluid concentrations were expressed in mass per volume units (ng/mL). All C-7 and AEDs concentrations below LLOQ were reported as zero in the concentration data listings. Concentrations below LLOQ were treated as zero in summary statistics of concentration data as well as for calculation of C-7 PK parameters. A geometric mean was not reported whenever at least one concentration was below the LLOQ (i.e. zero). Missing data were labeled as such and no imputation methods were used

Terminal elimination rate constant (lambda_z) was calculated as the slope of the linear regression of the terminal phase of the logarithmic concentration-time profile for each individual dataset available. A minimum of three time points was used for determining lambda_z. Regression was performed without weighting, and a minimum value of 0.75 for Rsq_adjusted was necessary for acceptance.

The different areas under the concentration-time curve were calculated using the linear trapezoidal summation (both ascending and descending phase).

Descriptive statistics of pharmacokinetic parameters included mean, SD, and CV, minimum and maximum. The geometric mean was identified when presented. A range of values were presented for selected variables. Since Tₘₐₓ is generally evaluated by a nonparametric method, median values and ranges were provided.

AED concentrations were listed and summarized; analysis of level of exposure of AED(s) taken by the patient was performed on an ad-hoc basis as it depended on specifics of the AED.

### 3.2 Representation of PK/PD in Relation to SPR

The existence of an exposure-response relationship was investigated graphically. A scatter plot of the area under the effect curve (AUECt, SPR change from baseline) versus area under the C-7 plasma concentrations curve (AUCt) was produced. A similar plot was produced for AUECt versus Cmax.

### 3.3 Suppression of PPR or Reduction of SPR

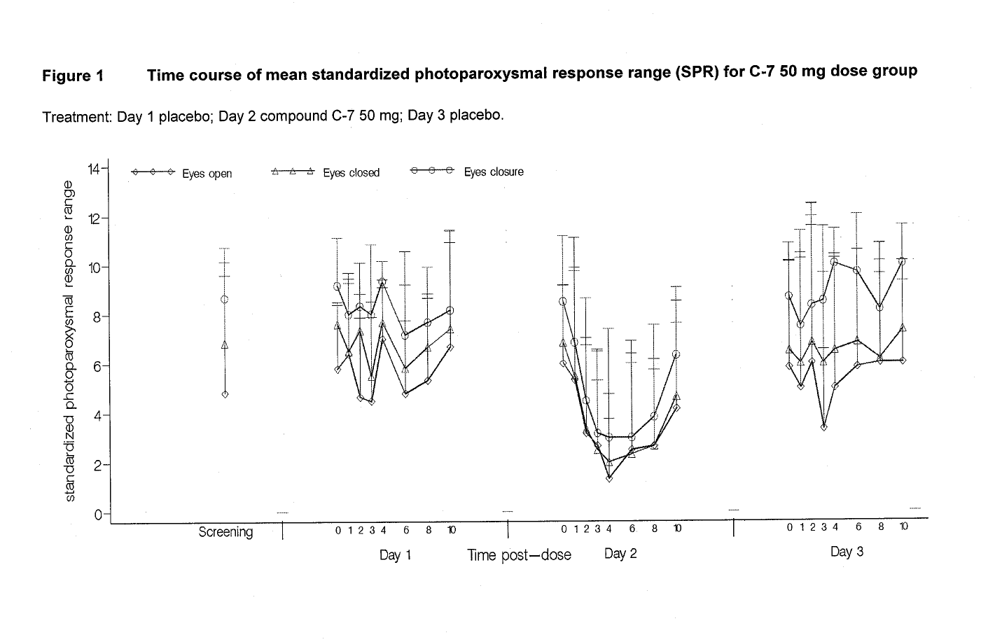

The SPR results for all eye conditions (i.e., eyes open, eyes closed or eyes closure) in Cohort I of the study are shown in FIG. 1. Interim analysis results of the C-7 50 mg dose group (Cohort I) showed that treatment with C-7 resulted in complete suppression of PPR in 3 of 6 patients and all 6 patients showed reductions of the SPR range by at least 3 steps in at least one eye condition on Day 2. In subsequent Cohorts, complete suppression of PPR was noted in 3 out of 4 patients in C-7 100 mg dose group (Cohort II) and 1 out of 3 patients in C-7 15 mg dose group (Cohort III); and all 7 patients (of Cohorts II and III) showed reductions of the SPR range by at least 3 steps in at least one eye condition on Day 2. The magnitude of the post-treatment effect differed between doses. Magnitude of effect was found dose dependent. Compound C-7 15 mg dose group showed a numerically markedly lower PD effect when compared to Compound C-7 50 and 100 mg dose groups.

Compound C-7 15 mg dose group showed a numerically lower but still measurable PD effect compared to 50 and 100 mg dose groups.

### 3.4 Time of Onset and Duration of Effect

The majority of patients had response onset within 1 to 2 hours of dosing. Time to maximum reduction of SPR was shorter in C-7 100 mg dose group compared to the other 2 dose groups.

The duration of response was similar in C-7 50 and 100 mg dose groups, ranging from 23 to 34 hours inclusive. Two patients in C-7 50 mg group and 1 patient in C-7 100 mg group maintained SPR response (i.e., stayed in responding status at all time points between first and last response), i.e. 33 hours in both patients. In the C-7 15 mg dose group, the response was noted to be intermittent in all eye conditions and none of the patients maintained the SPR response.

Suppression was observed in 7 of the 13 patients, with the onset ranging from 1 to 4 hours after dosing and the duration ranging from 2 to 32 hours. Of these 7 patients, 4 patients maintained suppression (i.e., stayed in suppression status at all time points between first and last suppression) that included 2 patients each in C-7 50 mg group and C-7 100 mg group, i.e. 2 hours, 7 hours, 4 hours and 3 hours respectively. One patient in the C-7 15 mg group showed onset of suppression at 2 hours post dosing, however the effect was not maintained and was noted only in one eye condition (eye open).

Overall, in the C-7 15 mg group, neither the SPR response nor PPR suppression was maintained.

The proportion of subjects with suppression of PPR was numerically lower in C-7 15 mg dose group (33%) compared to the C-7 50 mg (50%) and 100 mg (75%) dose groups.

### 3.5 Maximal Reduction in SPR

A summary of maximum reduction of SPR by eye condition and treatment is shown in Table 4. The maximum reduction of SPR by eye condition and treatment was observed on active treatment Day 2 in patients who received the highest dose (Cohort II, C-7 100 mg), and it was noted for eye closure condition.

TABLE 4
Summary of maximum standardized photoparoxysmal response
range (SPR) by eye and treatment conditions.

| Treatment | Eye condition | | Overall reduction | Reduction from predose Day 2 |
|---|---|---|---|---|
| C-7 50 mg | Eyes open | n | 6 | 6 |
| | | Mean (SD) | 6.8 (2.99) | 4.8 (3.43) |
| | | Median | 7.5 | 6.5 |
| | Eye closed | n | 6 | 6 |
| | | Mean (SD) | 6.0 (2.37) | 5.0 (2.00) |
| | | Median | 6.0 | 5.0 |
| | Eye closure | n | 6 | 6 |
| | | Mean (SD) | 7.7 (3.01) | 6.5 (2.95) |
| | | Median | 8.5 | 7.5 |
| C-7 100 mg | Eyes open | n | 4 | 4 |
| | | Mean (SD) | 8.8 (2.50) | 7.0 (1.41) |
| | | Median | 8.5 | 6.5 |
| | Eye closed | n | 4 | 4 |
| | | Mean (SD) | 7.5 (2.38) | 6.5 (2.38) |
| | | Median | 8.5 | 6.5 |
| | Eye closure | n | 4 | 4 |
| | | Mean (SD) | 9.3 (2.22) | 8.3 (1.50) |
| | | Median | 10.0 | 9.0 |
| C-7 15 mg | Eyes open | n | 3 | 3 |
| | | Mean (SD) | 4.0 (3.46) | 3.0 (4.36) |
| | | Median | 2.0 | 1.0 |
| | Eye closed | n | 3 | 3 |
| | | Mean (SD) | 6.7 (1.15) | 5.0 (3.61) |
| | | Median | 6.0 | 6.0 |
| | Eye closure | n | 3 | 3 |
| | | Mean (SD) | 8.0 (1.73) | 7.3 (2.89) |
| | | Median | 9.0 | 9.0 |

Note:
Maximum reductions for each subject over all timepoints

### 3.6 Area Under SPR Curve

The summary of AUECₜ by eye condition, treatment and visit in the primary PD analysis set was measured. Statistical analysis of change from Day 1 in AUECₜ by eye condition in the primary PD analysis set was measured.

### 3.7 Pharmacokinetics Results (PK Results)

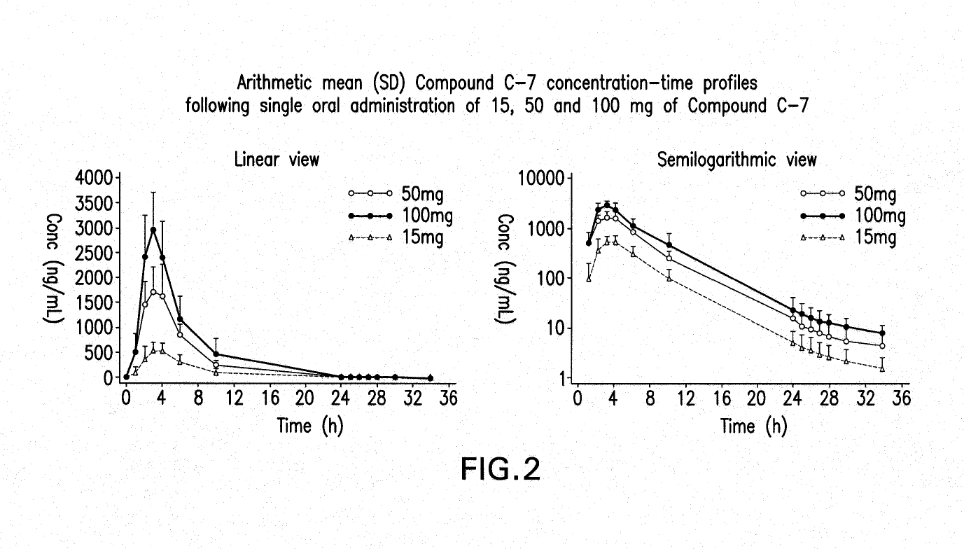

The arithmetic mean plasma concentration time profile of C-7 following a single oral administration of 15 (Cohort III), 50 (Cohort I) and 100 (Cohort II) mg to male and female patients with PSE is presented in FIG. 2.

Table 5 summarizes the C-7 PK parameters after a single dose administration of 15, 50 and 100 mg to male and female patients with PSE.

The patients received the study medication in the morning between 8:00 h and 9:00 h, at least 30 minutes after a light breakfast had been completed.

In the single dose healthy volunteer study at same or similar dose levels of C-7, C-7 was measurable in the plasma as early as 0.25 h post-dose following single administration of a 15, 50 or 100 mg dose to patients with PSE. The plasma concentration of C-7 peaked at around 3 h post-dose (median) with minimum values of approximately 2 h and maximum values of 4 h in individual patients.

The pharmacokinetic data of C-7 in the patients with PSE in all 3 cohorts appeared to be overlapping with those in male healthy subjects observed in healthy volunteer study, although mean exposure in patients with PSE tended to be slightly higher. A comparison of the means demonstrates that in patients mean Cₘₐₓ was about 10-20% and mean AUCₗₐₛₜ was about 10-40% higher than in healthy male subjects.

The inter-subject variability in Cₘₐₓ and AUCₗₐₛₜ was low to moderate with % CV geometric mean between 9% and 27% for Cₘₐₓ and between 13% and 36% for AUCs, as observed at the same or similar dose levels in the healthy volunteer study.

TABLE 5
Main Compound C-7 plasma PK parameters following single
oral administration of 15, 50 and 100 mg of Compound C-7

| | Cohort I | Cohort II | Cohort III |
|---|---|---|---|
| | 50 mg | 100 mg | 15 mg |
| PK parameters | n = 6 | n = 4 | n = 3 |
| T_{lag} (hr)¹ | 0.00 (0.00-0.00) | 0.00 (0.00-0.00) | 0.00 (0.00-0.00) |
| Tₘₐₓ (hr)¹ | 2.96 (2.00-3.98) | 2.94 (2.00-3.00) | 2.98 (1.97-4.00) |
| Cₘₐₓ (ng/mL)² | 1987 [27] | 2976 [26] | 637 [9] |
| AUCₗₐₛₜ | 10701 [25] | 17011 [19] | 3665 [21] |
| (hr*ng/mL)² | | | |
| AUC_{0-24 hr} | 10628 [25] | 16883 [19] | 3638 [20] |
| (hr*ng/mL)² | | | |
| AUC_{0-6 hr} | 6791 [21] | 10186 [36] | 2130 [13] |
| (hr*ng/mL)² | | | |

¹Median (min-max);
²Geometric mean [% CV geo mean]

### 4. Drug Dose, Drug Concentration and Relationships to Response

### 4.1 PK-PD Relationship

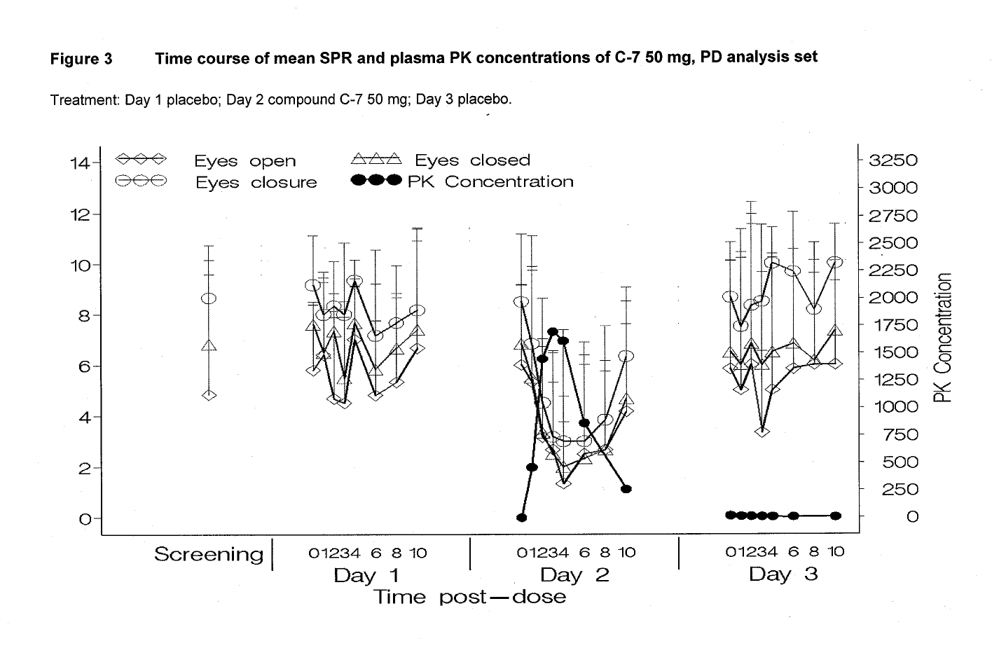

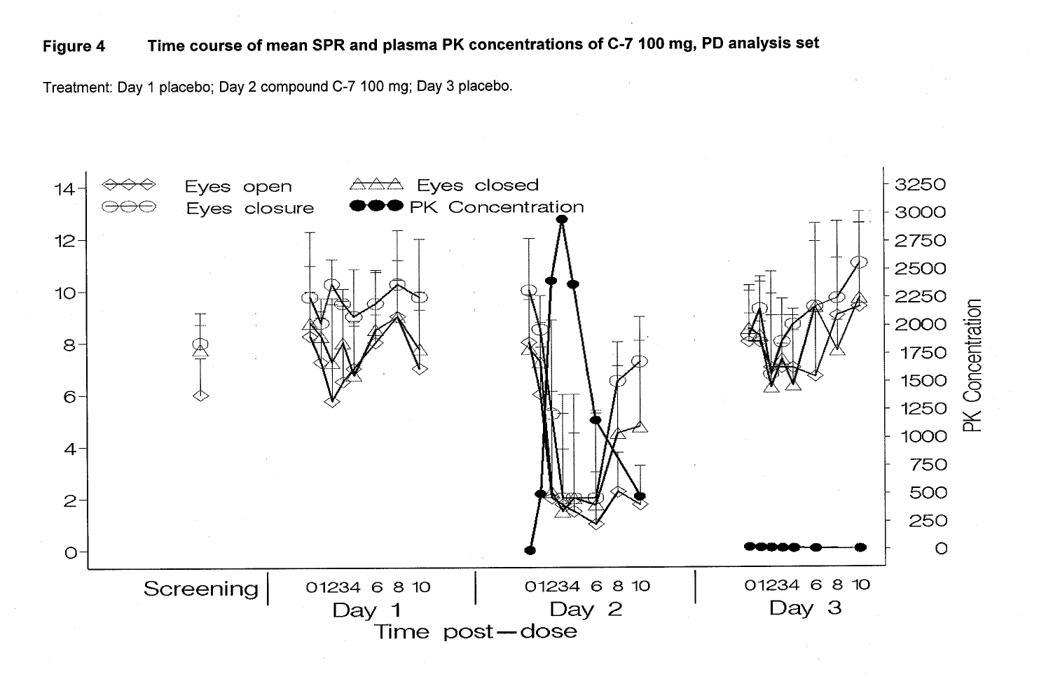

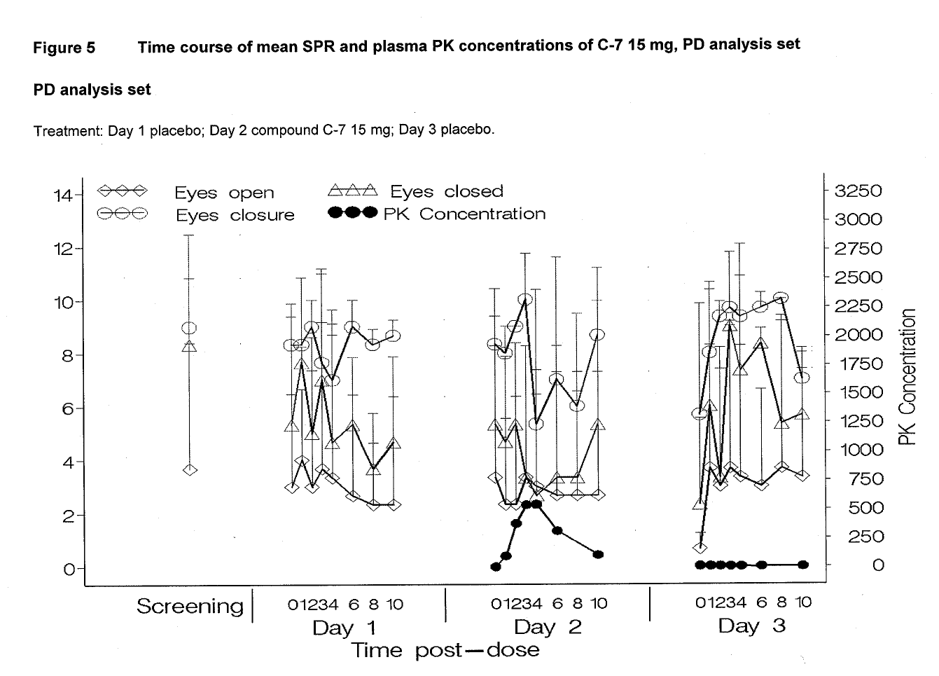

The mean SPR per eye condition and plasma PK concentrations of C-7 50, 100, and 15 mg dose groups is shown in FIG. 3, FIG. 4 and FIG. 5, respectively. The SPR and PK concentration over time plots show a linear relationship between the SPR Tₘₐₓ and PK Tₘₐₓ.

A PK-PD model was developed sequentially for the SPR data. Firstly a pharmacokinetic model was fitted using nonlinear mixed effects methodology in NONMEM version VI to describe the 13 patients with both PK and PD data. A 3 compartment disposition model with first order absorption and absorption lag time was selected to describe the pharmacokinetic time profile for each patient. The between patient variability was described by exponential random effects on each pharmacokinetic parameter, with all parameters assumed to be independent between individuals. The residual error model combined the additive and proportional error terms. The estimated pharmacokinetic parameters for each patient were used to generate each patient's PK profile as an input to the pharmacodynamic model.

For the Pharmacodynamic response, SPR, an Eₘₐₓ model was fitted on the logit transformed scale:

SPR=14/(1+exp[−{Base−Eₘₐₓ×C/(EC₅₀+C)}])

where Base is the baseline SPR on the logit scale, Eₘₐₓ is the maximum reduction in SPR on the logit scale, EC₅₀ is the concentration at which the 50% of the maximum reduction is obtained and C is C-7 concentration. For each patient, the PD parameters for the three eye conditions, eyes closure, eyes closed and eyes open, were estimated separately but with a common additive residual variance on the logit scale. The PD parameter estimates were highly variable across the 13 patients, with the estimation having trouble identifying Eₘₐₓ and EC₅₀ (see Table 6 below). The mean (SD) EC₅₀ for eyes open, eyes closed and eyes closures was 2964 ng/ml (4235 ng/ml), 3746 ng/ml (4475 ng/ml), 2252 ng/ml (3505 ng/ml) respectively. This wide variability was the result of either many patients having either a low estimated EC₅₀ (5 ng/ml) or high estimated EC₅₀ (9900 ng/ml). The mean (SD) Eₘₐₓ on the logit scale for eyes open, eyes closed and eyes closures was 21.6 (23.5), 16.3 (21.7), 12.9 (19.6) respectively suggesting there was a substantial reduction in SPR in the 3 eye conditions.

TABLE 6
Pharmacodynamic model parameter estimates for each patient

| | Eyes open | | | Eyes closed | | | Eyes closure | | |
|---|---|---|---|---|---|---|---|---|---|
| | Base | Eₘₐₓ | EC₅₀ | Base | Eₘₐₓ | EC₅₀ | Base | Eₘₐₓ | EC₅₀ |
| Patient A | — | — | — | — | — | — | 1.14 | 7.67 | 1410 |
| Patient B | −0.072 | 2.66 | 9900 | 0.406 | 0.487 | 5 | 0.425 | 0.05 | 5 |
| Patient G | 6.47 | 49.5 | 1880 | 6.38 | 49.5 | 2080 | 6.46 | 49.5 | 3080 |
| Patient M | −3.28 | 0.05 | 5 | −0.21 | 49.5 | 9200 | 0.321 | 0.495 | 83.3 |
| Patient C | 0.674 | 1.14 | 1470 | 0.538 | 0.963 | 219 | 1.16 | 2.38 | 1690 |
| Patient D | −0.144 | 40.8 | 9900 | −0.802 | 2.54 | 5 | 1.5 | 41.5 | 9900 |
| Patient E | 0.531 | 1.03 | 629 | 0.303 | 1.79 | 1170 | 0.164 | 0.571 | 320 |
| Patient F | −6.31 | 49.5 | 131 | −8.37 | 2.47 | 310 | 7.53 | 49.5 | 788 |
| Patient H | 0.0576 | 14.5 | 9900 | 1.11 | 6.99 | 2040 | 1.15 | 3.25 | 1270 |
| Patient I | 0.876 | 1.29 | 5 | 0.494 | 0.58 | 229 | 2.56 | 2.39 | 5 |
| Patient N | 0.0687 | 0.05 | 5 | 0.0692 | 0.05 | 9900 | 0.487 | 0.05 | 9900 |
| Patient O | 4.02 | 49.5 | 137 | −4.57 | 30.9 | 9900 | 4.69 | 9.23 | 10 |
| Patient L | 6.16 | 49.5 | 1610 | −4.23 | 49.5 | 9900 | −5.45 | 1.31 | 814 |

Onset of PD effect was noted either an hour before or around the same time when maximum plasma concentrations were achieved. A list of patients with C-7 AUC₀₋₂₄ₕ, Cₘₐₓ, Tₘₐₓ and their PD effect is presented in Table 7. Across the three treatment groups, the maximum and minimum AUC₀₋₂₄ₕ between which suppression of PPR was noted to be maintained was 21302 hr*ng/mL (patient H; C-7 100 mg group) and 10056 hr*ng/mL (patient A C-7 50 mg group), respectively. Similarly, the maximum and minimum AUC0-24 h between which relevant reduction of SPR was noted to be maintained was 15334 hr*ng/mL (patient I; C-7 100 mg group) and 11624 hr*ng/mL (patient D; C-7 50 mg group), respectively

Similarly, across the three treatment groups, the Cₘₐₓ between which suppression of PPR was noted to be maintained was 3700 ng/mL. (patient H, C-7 100 mg group) and 1530 ng/mL (patient A; C-7 50 mg group), respectively; and the Cₘₐₓ, between which relevant reduction of SPR was noted to be maintained was 2490 ng/mL (patient E, C-7 50 mg group) and 2070 ng/mL (patient I; C-7 100 mg group), respectively.

TABLE 7
List of patients with Compound C-7 C_{max,} Tₘₐₓ and their PD effect

| | | | | | | |
|---|---|---|---|---|---|---|
| | | | | | Onset | |
| Center/ | AUC_{0-24 h} | Cₘₐₓ | Tₘₐₓ | | Time | Effect |
| Patient # | (hr * ng/mL) | (ng/mL) | (hr) | PD effect^{d} | (h) | maintained |

Cohort I, C-7 50 mg

| | | | | | | |
|---|---|---|---|---|---|---|
| Patient A^{a} | 10056 | 1530 | 3.87 | Suppression of PPR | 4 | Yes |
| | | | | Reduction of SPR (≧3) | 2 | No |
| Patient B | 7043 | 1410 | 2.00 | Reduction of SPR (≧3) | 1 | No |
| Patient C | 9838 | 1910 | 3.00 | Reduction of SPR (≧3) | 2 | No |
| Patient D | 11624 | 2140 | 2.00 | Suppression of PPR | 2 | No |
| | | | | Reduction of SPR (≧3) | 1 | Yes |
| Patient E^{b} | 12564 | 2490 | 2.92 | Suppression of PPR | 3 | Yes |
| | | | | Reduction of SPR (≧3) | 1 | Yes |
| Patient F^{c} | 14162 | 2800 | 3.98 | Suppression of PPR^{e} | — | No |
| | | | | Reduction of SPR (≧3) | — | No |

Cohort II, C-7 100 mg

| | | | | | | |
|---|---|---|---|---|---|---|
| Patient G^{a} | 17924 | 3470 | 3.00 | Suppression of PPR | 2 | Yes |
| | | | | Reduction of SPR (≧3) | 1 | No |
| Patient H^{b} | 21302 | 3700 | 2.88 | Suppression of PPR | 3 | Yes |
| | | | | Reduction of SPR (≧3) | 2 | No |
| Patient I | 15334 | 2070 | 3.00 | Reduction of SPR (≧3) | 1 | Yes |
| Patient L | 13877 | 2950 | 2.00 | Suppression of PPR | 2 | No |
| | | | | Reduction of SPR (≧3) | 1 | No |

Cohort III, C-7 15 mg

| | | | | | | |
|---|---|---|---|---|---|---|
| Patient M | 4274 | 581 | 4.00 | Suppression of PPR^{e} | — | No |
| | | | | Reduction of SPR (≧3) | — | No |
| Patient N | 2906 | 634 | 1.97 | Reduction of SPR (≧3) | 1 | No |
| Patient O^{c} | 3878 | 701 | 2.98 | Suppression of PPR | 2 | No |
| | | | | Reduction of SPR (≧3) | 1 | No |

^{a,b,c}Same patients enrolled in two cohorts.
^{d}All patients showed the PD effect of reduction of SPR (≧3) in at least one eye condition.
^{e}Suppression of PPR was noted already at “0” hours of dosing on Day 2

### 4.2 Drug-Drug and Drug-Disease Interactions

The pharmacokinetic data of C-7 in the patients with photosensitive epilepsy in all 3 cohorts appeared to be similar to those in male healthy subjects observed in healthy volunteer study; although mean exposure in patients with photosensitive epilepsy tended to be slightly higher (patients mean Cₘₐₓ was about 10-20% and mean AUCₗₐₛₜ about 10-40% higher than in healthy male subjects).

Concomitant treatment with AEDs did not appear to be causative for the, slightly higher exposure since it is a patient without pre-existing AED treatment (patient F) that showed the highest exposure in Cohort I.

Since patients received the study medication at least 30 minutes after a light breakfast had been completed, versus fasted administration in healthy volunteer study, the slightly higher C-7 exposure observed in that study may be due to an increased C-7 bioavailability in the presence of food.

The co-administration of C-7 on Day 2 did not seem to affect the PK of selected AEDs (preexisting treatment), since no difference in the PK profiles could be observed between Day 1 and Days 2 and 3.

### Summary of Pharmacodynamic and Pharmacokinetic Results

### Pharmacodynamic Results

Treatment with compound C-7 resulted in complete suppression of PPR in 7 of 13 patients: 3 of 6 patients in C-7 50 mg group; 3 of 4 patients in compound C-7 100 mg group; and 1 of 3 patients in compound C-7 15 mg group. All 13 patients showed reductions of the SPR range by at least 3 steps in at least one eye condition on Day 2, i.e all doses of compound C-7 showed some effect.
The majority of patients had response onset within 1 to 2 hours of dosing. Time to maximum reduction of SPR was shorter in compound C-7 100 mg dose group compared to the other 2 dose groups.
compound C-7 15 mg dose group showed a numerically lower PD effect compared to compound C-7 50 and 100 mg dose groups.
A maximum reduction of mean SPR was noted in the compound C-7 100 mg dose group, and at time points 3, 4 and 6 hours post-dose.
The SPR response and suppression of PPR was maintained in compound C-7 50 and 100 mg dose groups. The maximum duration of maintenance was 33 hours and 7 hours for SPR response and suppression of PPR, respectively. In the compound C-7 15 mg group, neither the SPR response nor suppression of PPR was maintained.
Within the dose groups, magnitude (reduction of SPR vs suppression of PPR) and maintenance of PD effect appeared to be greater in patients with numerically higher Cₘₐₓ.
In compound C-7 50 mg and 100 mg dose groups, for all eye conditions, significantly lower AUECₜ was noted on Day 2 compared to Day 1.
Treatment with selected concomitant AEDs did not appear to relevantly impact the PD effect of compound C-7, i.e. there was no sign for a relevant PD interaction between C-7 and these AEDs.

### Pharmacokinetic Results

The pharmacokinetic data of compound C-7 in the patients with photosensitive epilepsy in all 3 cohorts appeared to be overlapping with those in male healthy subjects observed in healthy volunteer study, although mean exposure in patients with photosensitive epilepsy tended to be slightly higher (10-20% for Cₘₐₓ, 10-40% for AUCₗₐₛₜ).
With the exception of two patients in Cohort I (patients C and F) and one patient in Cohort III (patient O), who did not take AEDs, patients had pre-existing treatment with 1 AED (lamotrigine, valproate or zonisamide) or 2 AEDs (levetiracetam and topiramate for patient M in Cohort III). It was noted that patient C of Cohort I also participated in Cohort III (patient I), and therefore in total only 2 patients did not have prior concomitant AEDs in the study. Similar AEDs concentration-time profiles on Days 1, 2 and 3 for each patient confirmed stable pre-existing AED treatment. Co-administration of compound C-7 did not appear to affect the AED PK.

### PK-PD Results

SPR and PK concentration overtime plots show a linear relationship between SPR Tₘₐₓ and PK Tₘₐₓ.
The maximum and minimum AUC₀₋₂₄ₕ between which suppression of PPR was noted to be maintained was 21302 hr*ng/mL (patient H; C-7 100 mg group) and 10056 hr*ng/mL (patient A; C-7 50 mg group), respectively. Similarly, the maximum and minimum AUC₀₋₂₄ₕ between which relevant reduction of SPR (≧3 steps) was noted to be maintained was 15334 hr*ng/mL (patient I; C-7 100 mg group) and 11624 hr*ng/mL (patient D; C-7 50 mg group), respectively
The maximum and minimum Cₘₐₓ between which suppression of PPR was noted to be maintained was 3700 ng/mL (patient H, C-7 100 mg group) and 1530 ng/mL (patient A; C-7 50 mg group), respectively. Similarly, the maximum and minimum Cₘₐₓ between which relevant reduction of SPR was noted to be maintained was 2490 ng/mL (patient E, C-7 50 mg group) and 2070 ng/mL (patient I; C-7 100 mg group), respectively.

### Oral Bioavailability of the Compounds of the Invention

Oral bioavailability of the compounds of the invention may be demonstrated using any generally known test in which the compound is administered orally and a biological effect observed.

Oral bioavailability of the compounds of the invention in the treatment of photosensitive epilepsy may be further quantified by the Maximal Electroshock test, which demonstrates that the compounds are orally bioavailable, penetrate the blood brain barrier and bind to the target receptor.

The oral bioavailability was tested using the audiogenic mouse test (Audiogenic seizures, R. L. Collins; Chapter 14, pages: 347-372. In Experimental Models of Epilepsy; By Pupura, Penry, Tower, Woodbury, Walter, Raven Press, New York, 1972. Standard Book Number 0-911216-26-X) and/or the MES test Where the MES test was used (as described below), the result is given in Table 8.

### Table 8: In-Vivo Activity of Parent Compounds and Prodrugs in the Murine Maximal Electra Shock Test

Compounds of the invention were tested in OF1 mice using the maximal electroshock test (MES Test) described in detail by Schmutz et al., Naunyn-Schmiedeberg's Arch Pharmacol 1990, 342, 61-66. Briefly, generalized tonic-clonic convulsions of the hind extremities were induced by passing electrical current through temporal electrodes (50 Hz, 18 mA, 0.2 s). Mice treated by vehicle showed mean seizure durations of 12-14 s. 30 mg/kg carbamazepine was used as a positive control; mice were classified as protected by a compound if the duration of the seizure lasted only 3 second or less. Five mice were used for each treatment condition and the percentage of protected mice was used as readout (i.e. a compound could give 0%, 20%, 40%, 60%, 80% or 100% protection). Compounds of the invention were given at a dose of 50 mg/kg, p.o., 1 hour prior to induction of convulsions (i.e. “pre-treatment time—1 h”).

ED50 values (ED: effective dose) were calculated using GraphPad Prism, v4.02.

15 s after shock administration, mouse blood was collected for determination of compounds' blood exposure.

The results are shown below in Table 8.

TABLE 8

| | | In | MES-Test | |
|---|---|---|---|---|
| | | vivo | (1 h, po) | |
| | | orally | ED50 [mg/ | |
| Compound | Structure | active | kg] | IUPAC name |
| A-1 | 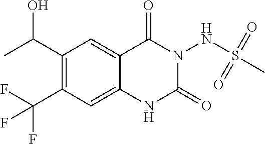 [SMILES: CC(O)c1cc2c(=O)n(NS(C)(=O)=O)c(=O)[nH]c2cc1C(F)(F)F] | Yes | 64 | N-[6-(1-Hydroxy-ethyl)-2,4-dioxo- 7-trifluoromethyl-1,4-dihydro-2H- quinazolin-3-yl]- methanesulfonamide |
| A-2 | 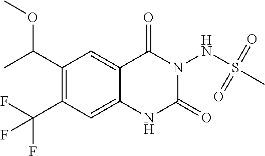 [SMILES: COC(C)c1cc2c(=O)n(NS(C)(=O)=O)c(=O)[nH]c2cc1C(F)(F)F] | Yes | 6.0 | N-[6-(1-Methoxy-ethyl)-2,4-dioxo- 7-trifluoromethyl-1,4-dihydro-2H- quinazolin-3-yl]- methanesulfonamide |
| A-3 | 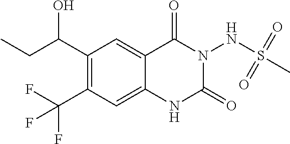 [SMILES: CCC(O)c1cc2c(=O)n(NS(C)(=O)=O)c(=O)[nH]c2cc1C(F)(F)F] | Yes | 19.6 | N-[6-(1-Hydroxy-propyl)-2,4-dioxo- 7-trifluoromethyl-1,4-dihydro-2H- quinazolin-3-yl]- methanesulfonamide |
| A-4 | 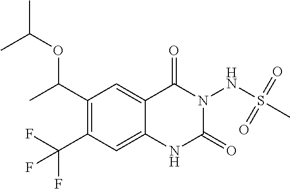 [SMILES: CC(C)OC(C)c1cc2c(=O)n(NS(C)(=O)=O)c(=O)[nH]c2cc1C(F)(F)F] | Yes | 15.6 | N-[6-(1-Isopropyl-ethyl)-2,4- dioxo-7-trifluoromethyl-1,4- dihydro-2H-quinazolin-3-yl]- methanesulfonamide |
| A-5 | 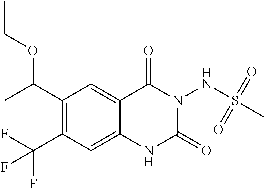 [SMILES: CCOC(C)c1cc2c(=O)n(NS(C)(=O)=O)c(=O)[nH]c2cc1C(F)(F)F] | Yes | 8.8 | N-[6-(1-Ethoxy-ethyl)-2,4-dioxo-7- trifluoromethyl-1,4-dihydro-2H- quinazolin-3-yl]- methanesulfonamide |
| A-6 | 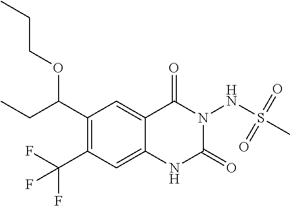 [SMILES: CCCOC(CC)c1cc2c(=O)n(NS(C)(=O)=O)c(=O)[nH]c2cc1C(F)(F)F] | Yes | nt¹ | N-[2,4-Dioxo-6-(1-propoxy- propyl)-7-trifluoromethyl-1,4- dihydro-2H-quinazolin-3-yl]- methanesulfonamide |
| A-7 | 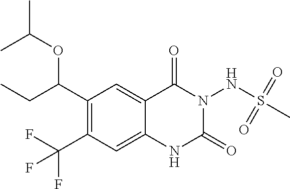 [SMILES: CCC(OC(C)C)c1cc2c(=O)n(NS(C)(=O)=O)c(=O)[nH]c2cc1C(F)(F)F] | Yes | 24.7 | N-[6-(1-isopropoxy-propyl)-2,4- dioxo-7-trifluoromethyl-1,4- dihydro-2H-quinazolin-3-yl]- methanesulfonamide |
| A-8 | 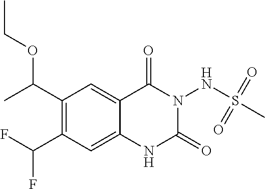 [SMILES: CCOC(C)c1cc2c(=O)n(NS(C)(=O)=O)c(=O)[nH]c2cc1C(F)F] | nt | nt | N-[7-Difluoromethyl-6-(1-ethoxy- ethyl)-2,4-dioxo-1,4-dihydro-2H- quinazolin-3-yl]- methanesulfonamide |
| A-9 | 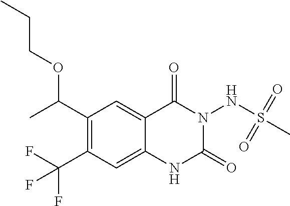 [SMILES: CCCOC(C)c1cc2c(=O)n(NS(C)(=O)=O)c(=O)[nH]c2cc1C(F)(F)F] | nt | nt | N-[2,4-Dioxo-6-(1-propoxy-ethyl)- 7-trifluoromethyl-1,4-dihydro-2H- quinazolin-3-yl]- methanesulfonamide |
| A-10 | 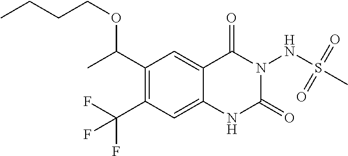 [SMILES: CCCCOC(C)c1cc2c(=O)n(NS(C)(=O)=O)c(=O)[nH]c2cc1C(F)(F)F] | nt | nt | N-[6-(1-Butoxy-ethyl)-2,4-dioxo-7- trifluoromethyl-1,4-dihydro-2H- quinazolin-3-yl]- methanesulfonamide |
| A-11 | 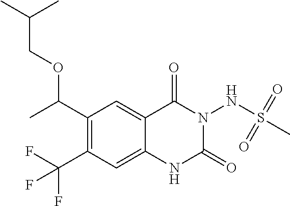 [SMILES: CC(C)COC(C)c1cc2c(=O)n(NS(C)(=O)=O)c(=O)[nH]c2cc1C(F)(F)F] | nt | nt | N-[6-(1-Isobutoxy-ethyl)-2,4-dioxo- 7-trifluoromethyl-1,4-dihydro-2H- quinazolin-3-yl]- methanesulfonamide |
| A-12 | 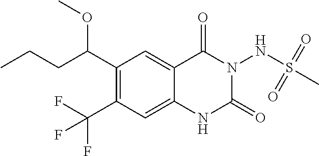 [SMILES: CCCC(OC)c1cc2c(=O)n(NS(C)(=O)=O)c(=O)[nH]c2cc1C(F)(F)F] | nt | nt | N-[6-(1-methoxy-butyl)-2,4-dioxo- 7-trifluoromethyl-1,4-dihydro-2H- quinazolin-3-yl]- methanesulfonamide |
| A-13 | 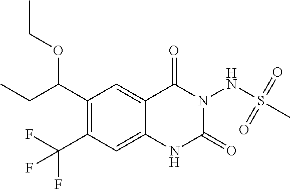 [SMILES: CCOC(CC)c1cc2c(=O)n(NS(C)(=O)=O)c(=O)[nH]c2cc1C(F)(F)F] | Yes | nt | N-[6-(1-Ethoxy-propyl)-2,4-dioxo- 7-trifluoromethyl-1,4-dihydro-2H- quinazolin-3-yl]- methanesulfonamide |
| A-14 | 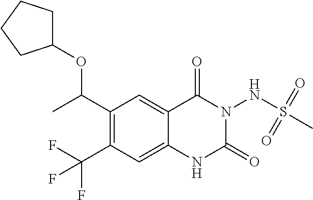 [SMILES: CC(OC1CCCC1)c1cc2c(=O)n(NS(C)(=O)=O)c(=O)[nH]c2cc1C(F)(F)F] | yes | nt | N-[6-(1-Cyclopentyloxy-ethyl)-2,4- dioxo-7-trifluoromethyl-1,4- dihydro-2H-quinazolin-3-yl]- methanesulfonamide |
| A-15 | 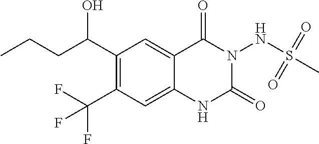 [SMILES: CCCC(O)c1cc2c(=O)n(NS(C)(=O)=O)c(=O)[nH]c2cc1C(F)(F)F] | Yes | 35 | N-[6-(1-Hydroxy-butyl)-2,4-dioxo- 7-trifluoromethyl-1,4-dihydro-2H- quinazolin-3-yl]- methanesulfonamide |
| A-16 | 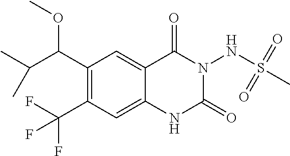 [SMILES: COC(c1cc2c(=O)n(NS(C)(=O)=O)c(=O)[nH]c2cc1C(F)(F)F)C(C)C] | nt | nt | N-[6-(1-Methoxy-2-methyl-propyl)- 2,4-dioxo-7-trifluoromethyl-1,4- dihydro-2H-quinazolin-3-yl]- methanesulfonamide |
| A-17 | 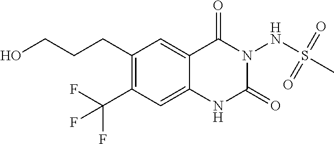 [SMILES: CS(=O)(=O)Nn1c(=O)[nH]c2cc(C(F)(F)F)c(CCCO)cc2c1=O] | nt | nt | N-[6-(3-Hydroxy-propyl)-2,4-dioxo- 7-trifluoromethyl-1,4-dihydro-2H- quinazolin-3-yl]- methanesulfonamide |
| A-18 | 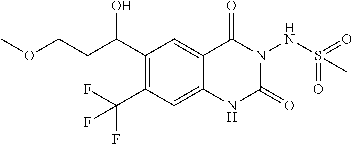 | Yes | nt | N-[6-(1-Hydroxy-3-methoxy- propyl)-2,4-dioxo-7- trifluoromethyl-1,4-dihydro-2H- quinazolin-3-yl]- methanesulfonamide |
| A-19 | 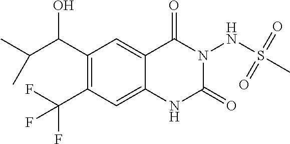 [SMILES: CC(C)C(O)c1cc2c(=O)n(NS(C)(=O)=O)c(=O)[nH]c2cc1C(F)(F)F] | nt | nt | N-[6-(1-Hydroxy-2-methyl-propyl)- 2,4-dioxo-7-trifluoromethyl-1,4- dihydro-2H-quinazolin-3-yl]- methanesulfonamide |
| B-1 | 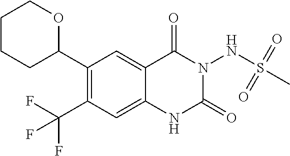 [SMILES: CS(=O)(=O)Nn1c(=O)[nH]c2cc(C(F)(F)F)c(C3CCCCO3)cc2c1=O] | nt | nt | N-[2-(4-Dioxo-6-(tetrahydro-pyran- 2-yl)-7-trifluoromethyl-1,4-dihydro- 2H-quinazolin-3-yl]- methanesulfonamide |
| B-2 | 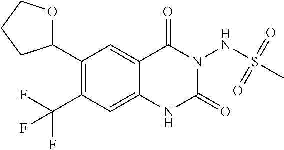 [SMILES: CS(=O)(=O)Nn1c(=O)[nH]c2cc(C(F)(F)F)c(C3CCCO3)cc2c1=O] | Yes | 12.8 (R)² 33.2 (S) | N-[2,4-Dioxo-6-(tetrahydro-furan- 2-yl)-7-trifluoromethyl-1,4-dihydro- 2H-quinazolin-3-yl]- methanesulfonamide |
| B-3 | 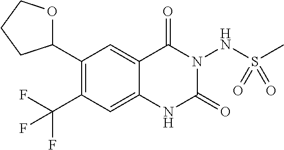 [SMILES: CS(=O)(=O)Nn1c(=O)[nH]c2cc(C(F)(F)F)c(C3CCCO3)cc2c1=O] | Yes | 20% @ 25³ | N-[2,4-Dioxo-6-(tetrahydro-furan- 3-yl)-7-trifluoromethyl-1,4-dihydro- 2H-quinazolin-3-yl]- methanesulfonamide |
| C-1 | 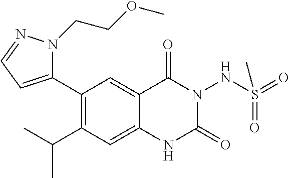 [SMILES: COCCn1nccc1-c1cc2c(=O)n(NS(C)(=O)=O)c(=O)[nH]c2cc1C(C)C] | yes | 40% @ 25 | N-{7-Isopropyl-6-[2-(2-methoxy- ethyl)-2H-pyrazol-3-yl]-2,4-dioxo- 1,4-dihydro-2H-quinazolin-3-yl}- methanesulfonamide |
| C-2 | 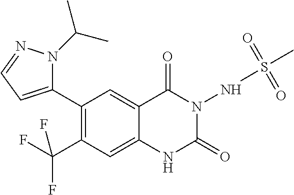 [SMILES: CC(C)n1nccc1-c1cc2c(=O)n(NS(C)(=O)=O)c(=O)[nH]c2cc1C(F)(F)F] | Yes | 17.7 | N-[6-(2-Isopropyl-2H-pyrazol-3-yl)- 2,4-dioxo-7-trifluoromethyl-1,4- dihydro-2H-quinazolin-3-yl]- methanesulfonamide |
| C-3 | 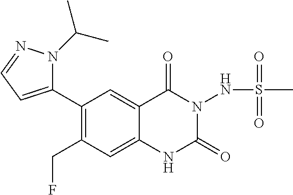 [SMILES: CC(C)n1nccc1-c1cc2c(=O)n(NS(C)(=O)=O)c(=O)[nH]c2cc1CF] | Yes | 13.5 | N-[7-Fluoromethyl-6-(2-isopropyl- 2H-pyrazol-3-yl)-2,4-dioxo-1,4- dihydro-2H-quinazolin-3-yl]- methanesulfonamide |
| C-4 | 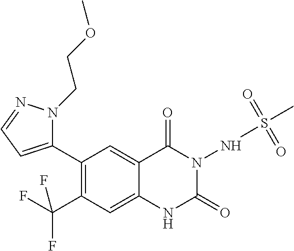 [SMILES: COCCn1nccc1-c1cc2c(=O)n(NS(C)(=O)=O)c(=O)[nH]c2cc1C(F)(F)F] | Yes | nt | N-{6-[2-(2-Methoxy-ethyl)-2H- pyrazol-3-yl]-2,4-dioxo-7- trifluoromethyl-1,4-dihydro-2H- quinazolin-3-yl}- methanesulfonamide |
| C-5 | 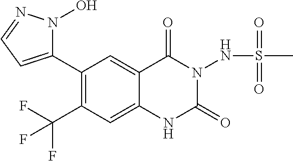 [SMILES: CS(=O)(=O)Nn1c(=O)[nH]c2cc(C(F)(F)F)c(-c3ccnn3O)cc2c1=O] | Yes | nt | N-[6-(2-Hydroxy-2H-pyrazol-3-yl)- 2,4-dioxo-7-trifluoromethyl-1,4- dihydro-2H-quinazolin-3-yl]- methanesulfonamide |
| C-6 | 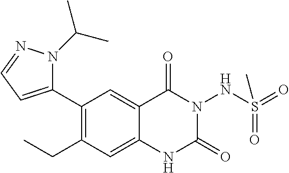 [SMILES: CCc1cc2[nH]c(=O)n(NS(C)(=O)=O)c(=O)c2cc1-c1ccnn1C(C)C] | yes | 20% @ 50 | N-[7-Ethyl-6-(2-isopropyl-2H- pyrazol-3-yl)-2,4-dioxo-1,4- dihydro-2H-quinazolin-3-yl]- methanesulfonamide |
| C-7 | 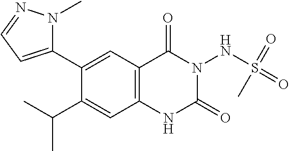 [SMILES: CC(C)c1cc2[nH]c(=O)n(NS(C)(=O)=O)c(=O)c2cc1-c1ccnn1C] | Yes | 6.9 | N-[7-Isopropyl-6-(2-methyl-2H- pyrazol-3-yl)-2,4-dioxo-1,4- dihydro-2H-quinazolin-3-yl]- methanesulfonamide |
| C-8 | 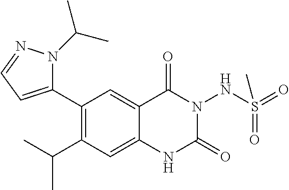 [SMILES: CC(C)c1cc2[nH]c(=O)n(NS(C)(=O)=O)c(=O)c2cc1-c1ccnn1C(C)C] | yes | 40% @ 50 | N-[7-Isopropyl-6-(2-isopropyl-2H- pyrazol-3-yl)-2,4-dioxo-1,4- dihydro-2H-quinazolin-3-yl]- methanesulfonamide |
| C-9 | 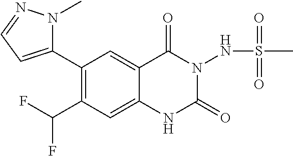 [SMILES: Cn1nccc1-c1cc2c(=O)n(NS(C)(=O)=O)c(=O)[nH]c2cc1C(F)F] | Yes | 7.5 | N-[7-Difluoromethyl-6-(2-methyl- 2H-pyrazol-3-yl)-2,4-dioxo-1,4- dihydro-2H-quinazolin-3-yl]- methanesulfonamide |
| C-10 | 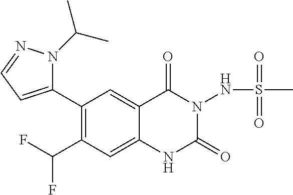 [SMILES: CC(C)n1nccc1-c1cc2c(=O)n(NS(C)(=O)=O)c(=O)[nH]c2cc1C(F)F] | Yes | 20.3 | N-[7-Difluoromethyl-6-(2- isopropyl-2H-pyrazol-3-yl)-2,4- dioxo-1,4-dihydro-2H-quinazolin- 3-yl]-methanesulfonamide |
| C-11 | 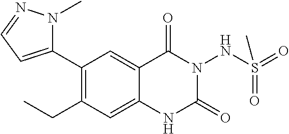 [SMILES: CCc1cc2[nH]c(=O)n(NS(C)(=O)=O)c(=O)c2cc1-c1ccnn1C] | Yes | 6.1 | N-[7-Ethyl-6-(2-methyl-2H-pyrazol- 3-yl)-2,4-dioxo-1,4-dihydro-2H- quinazolin-3-yl]- methanesulfonamide |
| C-12 | 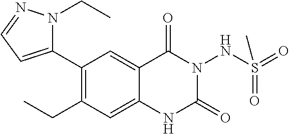 [SMILES: CCc1cc2[nH]c(=O)n(NS(C)(=O)=O)c(=O)c2cc1-c1ccnn1CC] | Yes | 42.8 | N-[7-Ethyl-6-(2-ethyl-2H-pyrazol- 3-yl)-2,4-dioxo-1,4-dihydro-2H- quinazolin-3-yl]- methanesulfonamide |
| C-13 | 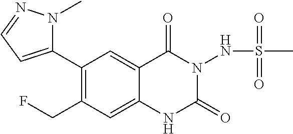 [SMILES: Cn1nccc1-c1cc2c(=O)n(NS(C)(=O)=O)c(=O)[nH]c2cc1CF] | nt | nt | N-[7-Fluoromethyl-6-(2-methyl-2H- pyrazol-3-yl)-2,4-dioxo-1,4- dihydro-2H-quinazolin-3-yl]- methanesulfonamide |
| C-14 | 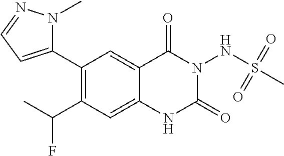 [SMILES: CC(F)c1cc2[nH]c(=O)n(NS(C)(=O)=O)c(=O)c2cc1-c1ccnn1C] | nt | nt | N-[7-(1-fluoro-ethyl)-6-(2-methyl- 2H-pyrazol-3-yl)-2,4-dioxo-1,4- dihydro-2H-quinazolin-3-yl]- methanesulfonamide |
| C-15 | 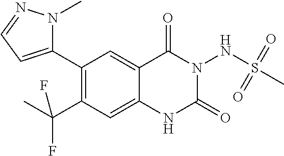 [SMILES: Cn1nccc1-c1cc2c(=O)n(NS(C)(=O)=O)c(=O)[nH]c2cc1C(C)(F)F] | Yes | 80% @ 20 | N-[7-(1,1-difluoro-ethyl)-6-(2- methyl-2H-pyrazol-3-yl)-2,4-dioxo- 1,4-dihydro-2H-quinazolin-3-yl]- methanesulfonamide |
| C-16 | 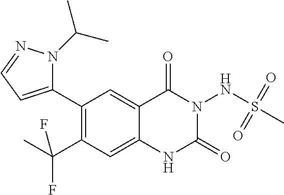 [SMILES: CC(C)n1nccc1-c1cc2c(=O)n(NS(C)(=O)=O)c(=O)[nH]c2cc1C(C)(F)F] | Yes | nt | N-[7-(1,1-difluoro-ethyl)-6-(2- isopropyl-2H-pyrazol-3-yl)-2,4- dioxo-1,4-dihydro-2H-quinazolin- 3-yl]-methanesulfonamide |
| C-17 | 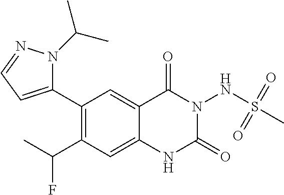 [SMILES: CC(F)c1cc2[nH]c(=O)n(NS(C)(=O)=O)c(=O)c2cc1-c1ccnn1C(C)C] | Yes | >20 | N-[7-(1-fluoro-ethyl)-6-(2- isopropyl-2H-pyrazol-3-yl)-2,4- dioxo-1,4-dihydro-2H-quinazolin- 3-yl]-methanesulfonamide |
| C-18 | 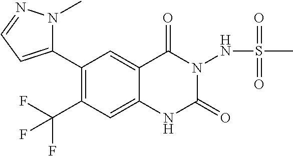 [SMILES: Cn1nccc1-c1cc2c(=O)n(NS(C)(=O)=O)c(=O)[nH]c2cc1C(F)(F)F] | Yes | 14.8 | N-[6-(2-Methyl-2H-pyrazol-3-yl)- 2,4-dioxo-7-trifluoromethyl-1,4- dihydro-2H-quinazolin-3-yl]- methanesulfonamide |
| Comparative | 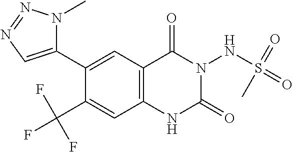 [SMILES: Cn1nncc1-c1cc2c(=O)n(NS(C)(=O)=O)c(=O)[nH]c2cc1C(F)(F)F] | No | 0% @ 50 | N-(6-(1-methyl-1H-1,2,3-triazol-5- yl)-2,4-dioxo-7-trifluoromethyl- 1,4-dihydro-2H-quinazolin-3-yl]- methanesulfonamide |

¹The term “nt” throughout the table means “not tested”
²(R) and (S) indicate the two enantiomers.
³The term “20% @ 25” means 20% protection at 20 mg/kg.

This data shows that the compounds for use in the invention exhibit beneficial oral bioavailability relating to the comparative example (not in accordance with the invention).

1. A compound of formula (I); 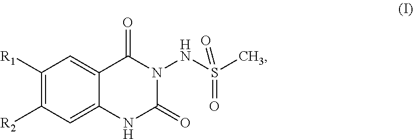
wherein
R₁ is C₁-C₆alkyl substituted by one, two or three substituents selected from hydroxy, C₁-C₆alkoxy or C₅-C₆cycloalkoxy; C₅-C₆cycloalkyl substituted by one, two or three substituents selected from hydroxy, C₁-C₆alkoxy or C₅-C₆cycloalkoxy; or
R₁ is 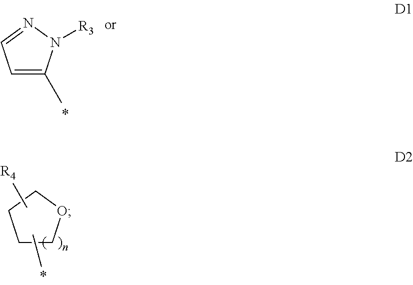
R₃ is C₁-C₆alkyl, hydroxy or C₁-C₆alkoxy-C₁-C₆alkyl;
R₄ is hydrogen or C₁-C₆alkyl;
n is 1 or 2;
R₂ is C₁-C₃alkyl or C₁-C₃fluoroalkyl;
or a pharmaceutically acceptable salt or prodrug thereof;
for use in the treatment or prevention of photosensitive epilepsy.

2. A compound of formula (I) according to claim 1, wherein R₁ is D1 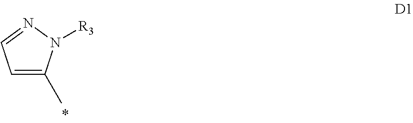
R₃ is C₁-C₆alkyl, hydroxy or C₁-C₆alkoxy-C₁-C₆alkyl;
R₂ is C₁-C₃alkyl or C₁-C₃fluoroalkyl;
or a pharmaceutically acceptable salt or prodrug thereof;
for use in the treatment or prevention of photosensitive epilepsy.

3. A compound of formula (I) according to claim 1, wherein R₁ is D2 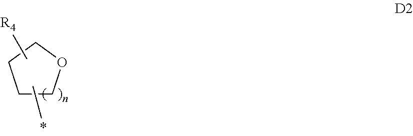
R₄ is hydrogen or C₁-C₆alkyl;
n is 1 or 2;
R₂ is C₁-C₃alkyl or C₁-C₃fluoroalkyl;
or a pharmaceutically acceptable salt or prodrug thereof;
for use in the treatment or prevention photosensitive epilepsy.

4. A compound of formula (I) for use according claim 1, selected from the group consisting of:
N-[6-(1-Hydroxy-ethyl)-2,4-dioxo-7-trifluoromethyl-1,4-dihydro-2H-quinazolin-3-yl]-methanesulfonamide;
N-[6-(1-Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[2,4-Dioxo-6-(1-propoxy-propyl)-7-trifluoromethyl-1,4-dihydro-2H-quinazolin-3-yl]-methanesulfonamide;
N-[6-(1 -isopropoxy-propyl)-2,4-dioxo-7-trifluoromethyl-1,4-dihydro-2H-quinazolin-3-yl]-methanesulfonamide;
N-[7-Difluoromethyl-6-(1-ethoxy-ethyl)-2,4-dioxo-1,4-dihydro-2H-quinazolin-3-yl]-methanesulfonamide;
N-[2,4-Dioxo-6-(1-propoxy-ethyl)-7-trifluoromethyl-1,4-dihydro-2H-quinazolin-3-yl]-methanesulfonamide;
N-[6-(1-Butoxy-ethyl)-2,4-dioxo-7-trifluoromethyl-1,4-dihydro-2H-quinazolin-3-yl]-methanesulfonamide;
N-[6-(1-Isobutoxy-ethyl)-2,4-dioxo-7-trifluoromethyl-1,4-dihydro-2H-quinazolin-3-yl]-methanesulfonamide;
N-[6-(1-methoxy-butyl)-2,4-dioxo-7-trifluoromethyl-1,4-dihydro-2H-quinazolin-3-yl]-methanesulfonamide;
N-[6-(1-Ethoxy-propyl)-2,4-dioxo-7-trifluoromethyl-1,4-dihydro-2H-quinazolin-3-yl]-methanesulfonamide;
N-[6-(1-Cyclopentyloxy-ethyl)-2,4-dioxo-7-trifluoromethyl-1,4-dihydro-2H-quinazolin-3-yl]-methanesulfonamide;
N-[6-(1-Hydroxy-butyl)-2,4-dioxo-7-trifluoromethyl-1,4-dihydro-2H-quinazolin-3-yl]-methanesulfonamide;
N-[6-(1-Methoxy-2-methyl-propyl)-2,4-dioxo-7-trifluoromethyl-1,4-dihydro-2H-quinazolin-3-yl]-methanesulfonamide;
N-[6-(3-Hydroxy-propyl)-2,4-dioxo-7-trifluoromethyl-1,4-dihydro-2H-quinazolin-3-yl]-methanesulfonamide;
N-[6-(1-Hydroxy-3-methoxy-propyl)-2,4-dioxo-7-trifluoromethyl-1,4-dihydro-2H-quinazolin-3-yl]-methanesulfonamide;
N-[6-(1-Hydroxy-2-methyl-propyl)-2,4-dioxo-7-trifluoromethyl-1,4-dihydro-2H-quinazolin-3-yl]-methanesulfonamide;
N-[2,4-Dioxo-6-(tetrahydro-pyran-2-yl)-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[2,4-Dioxo-6-(tetrahydro-furan-3-yl)-7-trifluoromethyl-1,4-dihydro-2H-quinazolin-3-yl]-methanesulfonamide;
N-{7-Isopropyl-6-[2-(2-methoxy-ethyl)-2H-pyrazol-3-yl]-2,4-dioxo-1,4-dihydro-2H-quinazolin-3-yl}-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-{6-[2-(2-Methoxy-ethyl)-2H-pyrazol-3-yl]-2,4-dioxo-7-trifluoromethyl-1,4-dihydro-2H-quinazolin-3-yl}-methanesulfonamide;
N-[6-(2-Hydroxy-2H-pyrazol-3-yl)-2,4-dioxo-7-trifluoromethyl-1,4-dihydro-2H-quinazolin-3-yl]-methanesulfonamide;
N-[7-Ethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-isopropyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-ethyl-2H-pyrazol-3-yl)-2,4-dioxo-1,4-dihydro-2H-quinazolin-3-yl]-methanesulfonamide;
N-[7-Fluoromethyl-6-(2-methyl-2H-pyrazol-3-yl)-2,4-dioxo-1,4-dihydro-2H-quinazolin-3-yl]-methanesulfonamide;
N-[7-(1-fluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-isopropyl-2H-pyrazol-3-yl)-2,4-dioxo-1,4-dihydro-2H-quinazolin-3-yl]-methanesulfonamide;
N-[7-(1-fluoro-ethyl)-6-(2-isopropyl-2H-pyrazol-3-yl)-2,4-dioxo-1,4-dihydro-2H-quinazolin-3-yl]-methanesulfonamide; or
N-[6-(2-Methyl-2H-pyrazol-3-yl)-2,4-dioxo-7-trifluoromethyl-1,4-dihydro-2H-quinazolin-3-yl]-methanesulfonamide;
or a pharmaceutically acceptable salt or prodrug thereof,
for use in the treatment or prevention photosensitive epilepsy.

5. A compound of formula (I) for use according to claim 1, selected from the group consisting of:
N-[6-(1 -Methoxy-ethyl)-2,4-dioxo-7-trifluoromethyl-1,4-dihydro-2H-quinazolin-3-yl]-methanesulfonamide;
N-[6-(1-Hydroxy-propyl)-2,4-dioxo-7-trifluoromethyl-1,4-dihydro-2H-quinazolin-3-yl]-methanesulfonamide;
N-[6-(1-Isopropoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[6-(2-Isopropyl-2H-pyrazol-3-yl)-2,4-dioxo-7-trifluoromethyl-1,4-dihydro-2H-quinazolin-3-yl]-methanesulfonamide;
N-[7-Fluoromethyl-6-(2-isopropyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
N-[7-(1,1-difluoro-ethyl)-6-(2-methyl-2H-pyrazol-3-yl)-2,4-dioxo-1,4-dihydro-2H-quinazolin-3-yl]-methanesulfonamide;
N-[6-(2-Methyl-2H-pyrazol-3-yl)-2,4-dioxo-7-trifluoromethyl-1,4-dihydro-2H-quinazolin-3-yl]-methanesulfonamide;
or a pharmaceutically acceptable salt or prodrug thereof,
for use in the treatment or prevention of photosensitive epilepsy.

6. A compound of formula (I) for use according to claim 1, selected from the group consisting of:
N-[6-(1-Methoxy-ethyl)-2,4-dioxo-7-trifluoromethyl-1,4-dihydro-2H-quinazolin-3-yl]-methanesulfonamide;
N-[6-(1-Ethoxy-ethyl)-2,4-dioxo-7-trifluoromethyl-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-methanesulfonamide;
N-[2,4-Dioxo-6-(tetrahydro-furan-2-yl)-7-trifluoromethyl-1,4-dihydro-2H-quinazolin-3-yl]-methanesulfonamide;
N-[7-Difluoromethyl-6-(2-methyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-methanesulfonamide;
or a pharmaceutically acceptable salt or prodrug thereof,
for use in the treatment or prevention of photosensitive epilepsy.

7. A compound of formula (I) for use according to claim 1, wherein the compound of formula (I) is N-[7-Isopropyl-6-(2-methyl-2H-pyrazol-3-yl)-2,4-dioxo-1,4-dihydro-2H-quinazolin-3-yl]-methanesulfonamide, or a pharmaceutically acceptable salt or prodrug thereof, for use in the treatment or prevention of photosensitive epilepsy.

8. A compound of formula (I) for use according to claim 1, wherein the compound of formula (I) is N-[7-Isopropyl-6-(2-methyl-2H-pyrazol-3-yl)-2,4-dioxo-1,4-dihydro-2H-quinazolin-3-yl]-methanesulfonamide.

9. A method for the treatment or prevention of photosensitive epilepsy in a subject in need of such treatment, which comprises administering to said subject a therapeutically effective amount of a compound of formula (I) as defined in claim 1 or a pharmaceutically acceptable salt or prodrug thereof.

10. A pharmaceutical composition comprising a compound of formula (I) as defined in claim 1 or a pharmaceutically acceptable salt or prodrug thereof, for use in the treatment or prevention of photosensitive epilepsy.

11. Use of a compound of formula (I) as defined in claim 1 or a pharmaceutically acceptable salt or prodrug thereof, in the manufacture of a medicament for use in the treatment or prevention of photosensitive epilepsy.

12. A compound of formula (I) according to claim 1 or a pharmaceutically acceptable salt or prodrug thereof in combination with one or more antiepileptic drugs (AEDs), for use in the treatment or prevention of photosensitive epilepsy.

13. A formulation comprising a compound of Formula (I) as defined in claim 1, wherein the compound comprised in the formulation gives a Cₘₐₓ of greater than or equal to 300 ng/mL.

14. A formulation according to claim 13, wherein the compound comprised in the formulation, additionally gives an AUC₂₄ₕ greater than or equal to 5000 mg*hr/mL.

15. A formulation according to claim 13, wherein the amount of the compound of formula (I) in the formulation is from 5 to 200 mg.

16. A formulation according to claim 13, wherein the compound of formula (I) is C-7.

17. A formulation according to claim 13 for use in the treatment or prevention of PSE.

18. A formulation comprising compound C7 for use in the treatment of PSE and having an AUC₂₄ₕ and/ or Cₘₐₓ such that the PPR is suppressed and/or the SPR is reduced, e.g. by at least 3 steps.
